(19) United States
(12) Patent Application Publication
(10) Pub. No.: US 2026/0093442 A1
(43) Pub. Date: Apr. 2, 2026

(54) AUDIO PLAYBACK SPEED ADJUSTMENT

(71) Applicant: QUALCOMM Incorporated, San Diego, CA (US)

(72) Inventors: Jason FILOS, Newark, NJ (US); Ricardo De Jesus BERNAL CASTILLO, San Diego, CA (US); Andre SCHEVCIW, San Diego, CA (US)

(21) Appl. No.: 19/114,997

(22) PCT Filed: Nov. 21, 2023
(86) PCT No.: PCT/US2023/080704

§ 371 Date: Mar. 25, 2025

(30) Foreign Application Priority Data
Dec. 9, 2022 (GR) 20220101019

(51) Int. Cl. G06F 3/16; A61B 5/00

(52) U.S. Cl. CPC G06F 3/165; A61B 5/4857

(57) ABSTRACT

A device includes one or more processors configured to obtain audio data with a first playback tempo. The one or more processors are also configured to receive biophysical sensor data indicative of a detected biophysical rhythm of a person. The one or more processors are further configured to adjust a playback speed of the audio data so that the audio data has a target playback tempo that matches a target biophysical rhythm. The target biophysical rhythm is based at least in part on the detected biophysical rhythm.

### I. CROSS-REFERENCE TO RELATED APPLICATIONS

The present application claims the benefit of priority from the commonly owned Greece Provisional Patent Application No. 20220101019, filed Dec. 9, 2022, the contents of which are expressly incorporated herein by reference in their entirety.

### II. FIELD The present disclosure is generally related to audio playback speed adjustment.

### III. DESCRIPTION OF RELATED ART

Advances in technology have resulted in smaller and more powerful computing devices. For example, there currently exist a variety of portable personal computing devices, including wireless telephones such as mobile and smart phones, tablets and laptop computers that are small, lightweight, and easily carried by users. These devices can communicate voice and data packets over wireless networks. Further, many such devices incorporate additional functionality such as a digital still camera, a digital video camera, a digital recorder, and an audio file player. Also, such devices can process executable instructions, including software applications, such as a web browser application, that can be used to access the Internet. As such, these devices can include significant computing capabilities.

Such computing devices often incorporate functionality to playback audio. Studies have shown that listening to music while exercising can improve performance. A person's level of exertion typically varies during an exercise session. Faster music is better suited to improve performance during a high energy middle portion of an exercise session, whereas slower music is better suited during a cool down portion at the end. Automatic adjustment of a playback tempo of audio can improve user experience.

### IV. SUMMARY

According to one implementation of the present disclosure, a device includes one or more processors configured to obtain audio data with a first playback tempo. The one or more processors are also configured to receive biophysical sensor data indicative of a detected biophysical rhythm of a person. The one or more processors are further configured to adjust a playback speed of the audio data so that the audio data has a target playback tempo that matches a target biophysical rhythm. The target biophysical rhythm is based at least in part on the detected biophysical rhythm.

According to another implementation of the present disclosure, a method includes obtaining, at a device, audio data with a first playback tempo. The method also includes receiving, at the device, biophysical sensor data indicative of a detected biophysical rhythm of a person. The method further includes adjusting, at the device, a playback speed of the audio data so that the audio data has a target playback tempo that matches a target biophysical rhythm, the target biophysical rhythm based at least in part on the detected biophysical rhythm.

According to another implementation of the present disclosure, a non-transitory computer-readable medium includes instructions that, when executed by one or more processors, cause the one or more processors to obtain audio data with a first playback tempo. The instructions, when executed by the one or more processors, also cause the one or more processors to receive biophysical sensor data indicative of a detected biophysical rhythm of a person. The instructions, when executed by the one or more processors, further cause the one or more processors to adjust a playback speed of the audio data so that the audio data has a target playback tempo that matches a target biophysical rhythm. The target biophysical rhythm is based at least in part on the detected biophysical rhythm.

According to another implementation of the present disclosure, an apparatus includes means for obtaining audio data with a first playback tempo. The apparatus also includes means for receiving biophysical sensor data indicative of a detected biophysical rhythm of a person. The apparatus further includes means for adjusting a playback speed of the audio data so that the audio data has a target playback tempo that matches a target biophysical rhythm. The target biophysical rhythm is based at least in part on the detected biophysical rhythm.

Other aspects, advantages, and features of the present disclosure will become apparent after review of the entire application, including the following sections: Brief Description of the Drawings, Detailed Description, and the Claims.

### V. BRIEF DESCRIPTION OF THE DRAWINGS

FIG. 1 is a block diagram of a particular illustrative aspect of a system operable to adjust audio playback speed, in accordance with some examples of the present disclosure.

FIG. 2 is a diagram of an illustrative aspect of operations associated with a rhythm estimator of the system of FIG. 1, in accordance with some examples of the present disclosure.

FIG. 3A is a diagram of an illustrative aspect of operations associated with a target predictor of the system of FIG. 1, in accordance with some examples of the present disclosure.

FIG. 3B is a diagram of another illustrative aspect of operations associated with a target predictor of the system of FIG. 1, in accordance with some examples of the present disclosure.

FIG. 4A is a diagram of an illustrative aspect of operations associated with a graph convolutional network (GCN) of the system of FIG. 1, in accordance with some examples of the present disclosure.

FIG. 4B is a diagram of another illustrative aspect of operations associated with a GCN of the system of FIG. 1, in accordance with some examples of the present disclosure.

FIG. 5A is a block diagram of an illustrative implementation of a system operable to adjust audio playback speed based on an estimated playback tempo, in accordance with some examples of the present disclosure.

FIG. 5B is a block diagram of an illustrative implementation of a system operable to adjust audio playback speed based on received tempo information, in accordance with some examples of the present disclosure.

FIG. 5C is a block diagram of an illustrative implementation of a system operable to adjust audio playback speed based on a detected mood, in accordance with some examples of the present disclosure.

FIG. 6 is a block diagram of an illustrative implementation of a system operable to adjust audio playback speed responsive to detection of a playback condition, in accordance with some examples of the present disclosure.

FIG. 7 is a block diagram of an illustrative implementation of a system operable to adjust audio playback speed based on updated biophysical sensor data, in accordance with some examples of the present disclosure.

FIG. 8 is a block diagram of an illustrative implementation of a system operable to adjust audio playback speed based on biophysical sensor data that satisfies a sensor data selection criterion, in accordance with some examples of the present disclosure.

FIG. 9A is a diagram of an illustrative aspect of operations associated with a rhythm combiner of the system of FIG. 1, in accordance with some examples of the present disclosure.

FIG. 9B is a diagram of another illustrative aspect of operations associated with a rhythm combiner of the system of FIG. 1, in accordance with some examples of the present disclosure.

FIG. 9C is a diagram of an illustrative aspect of operations associated with a tempo combiner of the system of FIG. 1, in accordance with some examples of the present disclosure.

FIG. 10A is a block diagram of an illustrative implementation of a system operable to adjust audio playback speed based at least in part on a target biophysical rhythm determined at another device, in accordance with some examples of the present disclosure.

FIG. 10B is a block diagram of an illustrative implementation of a system operable to adjust audio playback speed based on biophysical sensor data received from another device, in accordance with some examples of the present disclosure.

FIG. 11 is a diagram of an illustrative aspect of operation of components of a system operable to adjust audio playback speed, in accordance with some examples of the present disclosure.

FIG. 12 is a diagram of an illustrative aspect of operation of components of a system operable to adjust audio playback speed, in accordance with some examples of the present disclosure.

FIG. 13 illustrates an example of an integrated circuit operable to adjust audio playback speed, in accordance with some examples of the present disclosure.

FIG. 14 is a diagram of a mobile device operable to adjust audio playback speed, in accordance with some examples of the present disclosure.

FIG. 15 is a diagram of a headset operable to adjust audio playback speed, in accordance with some examples of the present disclosure.

FIG. 16 is a diagram of earbuds operable to adjust audio playback speed, in accordance with some examples of the present disclosure.

FIG. 17 is a diagram of a wearable electronic device operable to adjust audio playback speed, in accordance with some examples of the present disclosure.

FIG. 18 is a diagram of an extended reality glasses device operable to adjust audio playback speed, in accordance with some examples of the present disclosure.

FIG. 19 is a diagram of a voice-controlled speaker system operable to adjust audio playback speed, in accordance with some examples of the present disclosure.

FIG. 20 is a diagram of a headset, such as a virtual reality, mixed reality, or augmented reality headset, operable to adjust audio playback speed, in accordance with some examples of the present disclosure.

FIG. 21 is a diagram of a first example of a vehicle operable to adjust audio playback speed, in accordance with some examples of the present disclosure.

FIG. 22 is a diagram of a first example of a vehicle operable to adjust audio playback speed, in accordance with some examples of the present disclosure.

FIG. 23 is a diagram of a particular implementation of a method of audio playback speed adjustment that may be performed by the system of FIG. 1, in accordance with some examples of the present disclosure.

FIG. 24 is a block diagram of a particular illustrative example of a device that is operable to adjust audio playback speed, in accordance with some examples of the present disclosure.

### VI. DETAILED DESCRIPTION

Studies have shown that listening to music while exercising can improve performance. For example, a runner can cover greater distance while listening to higher tempo music. Automatic playback tempo adjustment of audio to match a target biophysical rhythm of a person listening to the audio can help the person reach and maintain the target biophysical rhythm. Illustrative non-limiting examples of a biophysical rhythm include a heartbeat (e.g., beats per minute), a gait cadence (e.g., steps per minute), a cycling cadence (e.g., pedal revolutions per minute), a swim cadence (e.g., strokes per minute), or a combination thereof.

Systems and methods of audio playback speed adjustment are disclosed. In an example, an audio analyzer receives biophysical sensor data from a sensor (e.g., a heart rate monitor, a camera, a pedometer, etc.). The biophysical sensor data is indicative of a detected biophysical rhythm of a person. The audio analyzer predicts a target biophysical rhythm of the person. The target biophysical rhythm (e.g., 130 steps per minute) can be based on the detected biophysical rhythm (e.g., 70 steps per minute) of the person, a user input, a detected exercise stage (e.g., starting a 30 minute workout), etc. The audio analyzer determines a target playback tempo (e.g., 130 beats per minute (BPM)) corresponding to the target biophysical rhythm (e.g., 130 steps per minute).

The audio analyzer determines a first playback tempo (e.g., a default tempo) of audio data that corresponds to a first playback speed (e.g., a default speed) of the audio data. The audio analyzer determines a target playback speed of the audio data based on a comparison of the first playback tempo (e.g., 120 BPM) and the target playback tempo (e.g., 130 BPM). For example, at the target playback speed (e.g., 130/120=108%), the audio data has the target playback tempo (e.g., 130 BPM). The audio analyzer adjusts a playback speed of the audio data to the target playback speed (e.g., 108%). The audio analyzer initiates playback, via a speaker, of an audio signal corresponding to the audio data having the adjusted playback speed (e.g., 108%).

In some examples, the audio signal corresponding to the audio data is already being output at a particular playback speed (e.g., the first playback speed or another adjusted playback speed) and the audio analyzer transitions from the particular playback speed to the adjusted playback speed (e.g., 130 BPM) over a time interval to avoid a sudden jump in playback speed.

In some examples, the audio analyzer selects a first target playback speed corresponding to the detected biophysical rhythm (e.g., 70 steps per minute) and a second target playback speed corresponding to the target biophysical rhythm (e.g., 130 steps per minute), and outputs the audio signal that transitions over a time interval from corresponding to audio data having the first target playback speed to audio data having the second target playback speed.

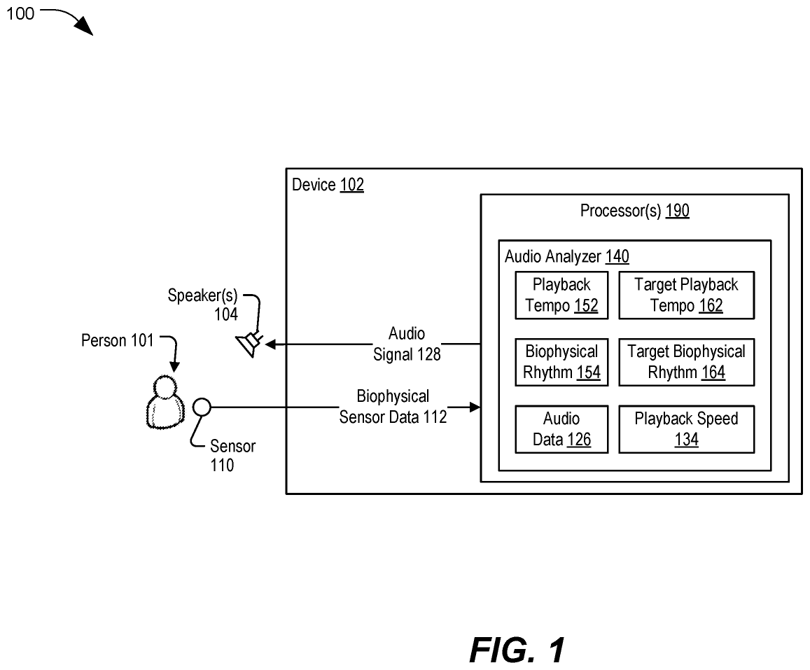

Particular aspects of the present disclosure are described below with reference to the drawings. In the description, common features are designated by common reference numbers. As used herein, various terminology is used for the purpose of describing particular implementations only and is not intended to be limiting of implementations. For example, the singular forms “a,” “an,” and “the” are intended to include the plural forms as well, unless the context clearly indicates otherwise. Further, some features described herein are singular in some implementations and plural in other implementations. To illustrate, FIG. 1 depicts a device 102 including one or more processors (“processor(s)” 190 of FIG. 1), which indicates that in some implementations the device 102 includes a single processor 190 and in other implementations the device 102 includes multiple processors 190. For ease of reference herein, such features are generally introduced as “one or more” features and are subsequently referred to in the singular unless aspects related to multiple of the features are being described.

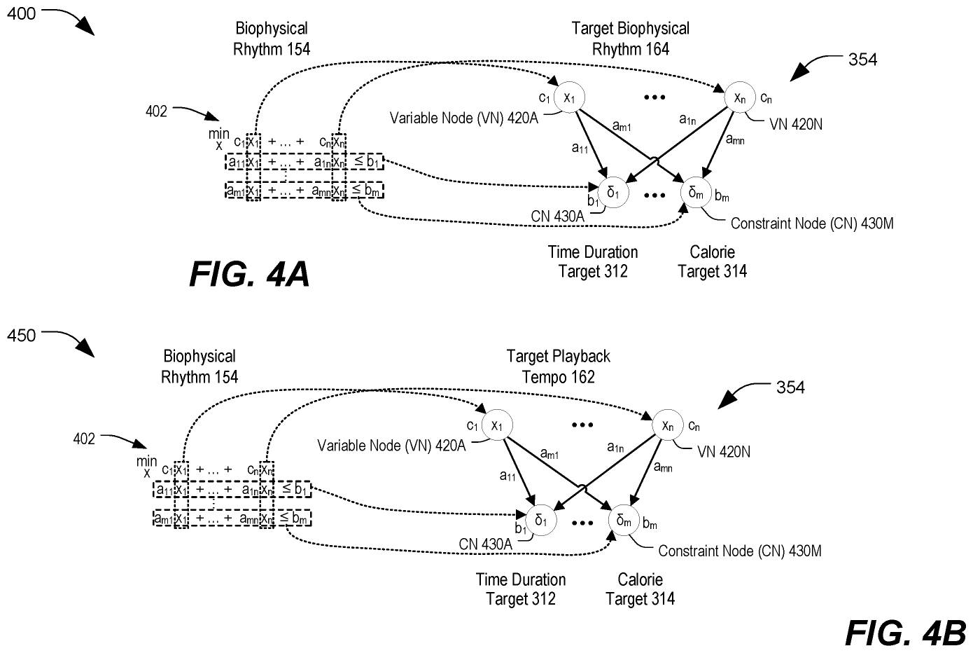

In some drawings, multiple instances of a particular type of feature are used. Although these features are physically and/or logically distinct, the same reference number is used for each, and the different instances are distinguished by addition of a letter to the reference number. When the features as a group or a type are referred to herein e.g., when no particular one of the features is being referenced, the reference number is used without a distinguishing letter. However, when one particular feature of multiple features of the same type is referred to herein, the reference number is used with the distinguishing letter. For example, referring to FIG. 4A, multiple variable nodes are illustrated and associated with reference numbers 420A and 420N. When referring to a particular one of these variable nodes, such as a variable node 420A, the distinguishing letter “A” is used. However, when referring to any arbitrary one of these variable nodes or to these variable nodes as a group, the reference number 420 is used without a distinguishing letter.

As used herein, the terms “comprise,” “comprises,” and “comprising” may be used interchangeably with “include,” “includes,” or “including.” Additionally, the term “wherein” may be used interchangeably with “where.” As used herein, “exemplary” indicates an example, an implementation, and/or an aspect, and should not be construed as limiting or as indicating a preference or a preferred implementation. As used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). As used herein, the term “set” refers to one or more of a particular element, and the term “plurality” refers to multiple (e.g., two or more) of a particular element.

As used herein, “coupled” may include “communicatively coupled,” “electrically coupled,” or “physically coupled,” and may also (or alternatively) include any combinations thereof. Two devices (or components) may be coupled (e.g., communicatively coupled, electrically coupled, or physically coupled) directly or indirectly via one or more other devices, components, wires, buses, networks (e.g., a wired network, a wireless network, or a combination thereof), etc. Two devices (or components) that are electrically coupled may be included in the same device or in different devices and may be connected via electronics, one or more connectors, or inductive coupling, as illustrative, non-limiting examples. In some implementations, two devices (or components) that are communicatively coupled, such as in electrical communication, may send and receive signals (e.g., digital signals or analog signals) directly or indirectly, via one or more wires, buses, networks, etc. As used herein, “directly coupled” may include two devices that are coupled (e.g., communicatively coupled, electrically coupled, or physically coupled) without intervening components.

In the present disclosure, terms such as “determining,” “calculating,” “estimating,” “shifting,” “adjusting,” etc. may be used to describe how one or more operations are performed. It should be noted that such terms are not to be construed as limiting and other techniques may be utilized to perform similar operations. Additionally, as referred to herein, “generating,” “calculating,” “estimating,” “using,” “selecting,” “accessing,” and “determining” may be used interchangeably. For example, “generating,” “calculating,” “estimating,” or “determining” a parameter (or a signal) may refer to actively generating, estimating, calculating, or determining the parameter (or the signal) or may refer to using, selecting, or accessing the parameter (or signal) that is already generated, such as by another component or device.

Referring to FIG. 1, a particular illustrative aspect of a system configured to adjust audio playback speed is disclosed and generally designated 100. The system 100 includes a device 102 that is coupled to one or more speakers 104 and to a sensor 110. In a particular aspect, the sensor 110 includes a heart rate monitor, a camera, a pedometer, another type of biophysical sensor, or a combination thereof.

The device 102 includes one or more processors 190 that include an audio analyzer 140 configured to perform audio playback speed adjustment. The audio analyzer 140 is configured to receive, from the sensor 110, biophysical sensor data 112 indicating a biophysical rhythm 154 of a person 101. The audio analyzer 140 is configured to adjust a playback speed 134 of audio data 126 based at least in part on the biophysical sensor data 112. In an example, the audio analyzer 140 is configured to determine a target playback tempo 162 based at least in part on the biophysical sensor data 112, as further described with reference to FIGS. 3A-3B. The audio analyzer 140 is configured to adjust the playback speed 134 so that the audio data 126 has the target playback tempo 162. The audio analyzer 140 is configured to output, to the one or more speakers 104, an audio signal 128 corresponding to the audio data 126 having the target playback tempo 162.

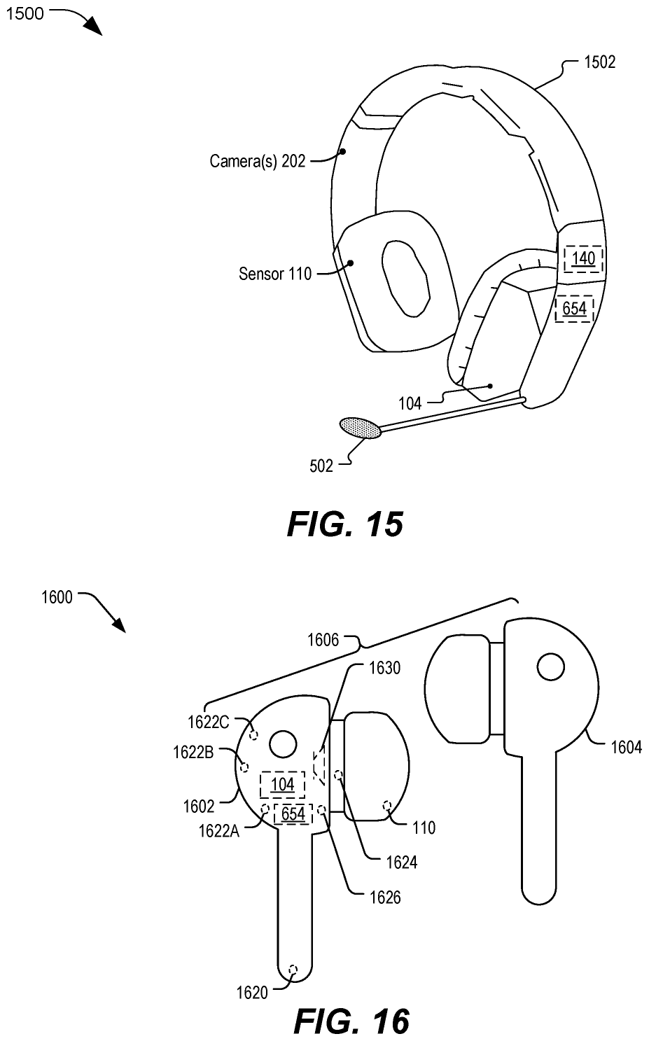

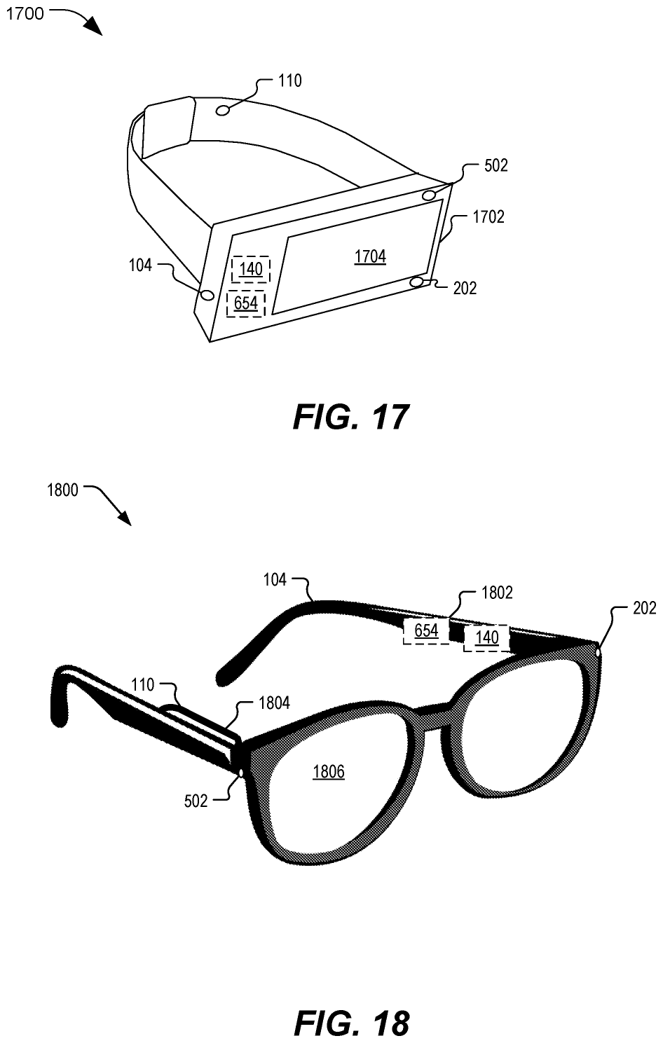

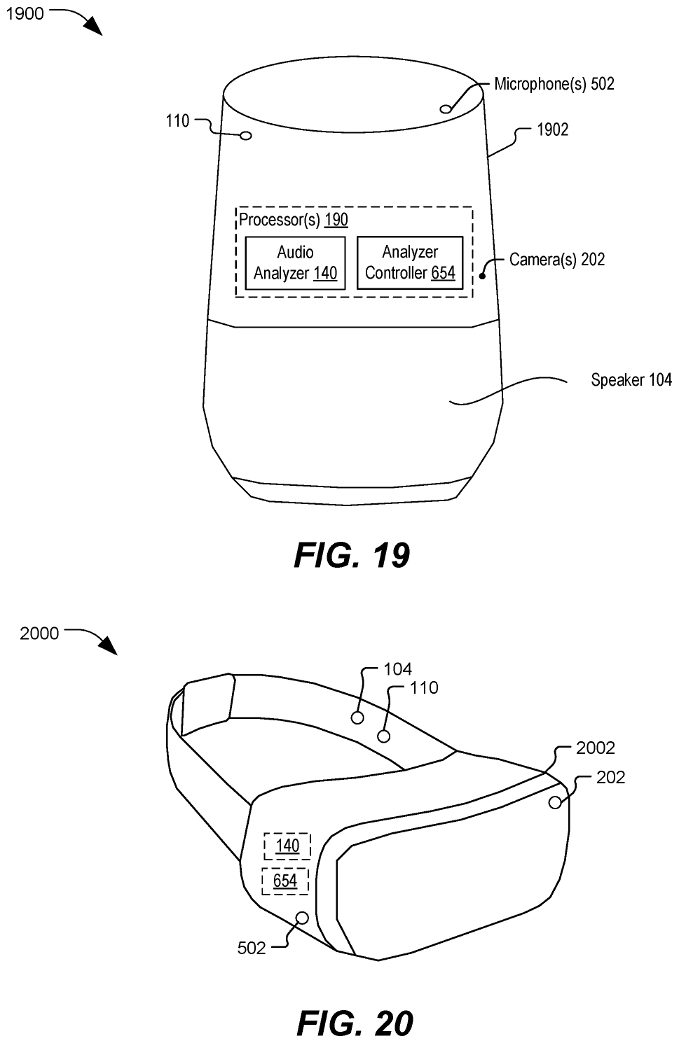

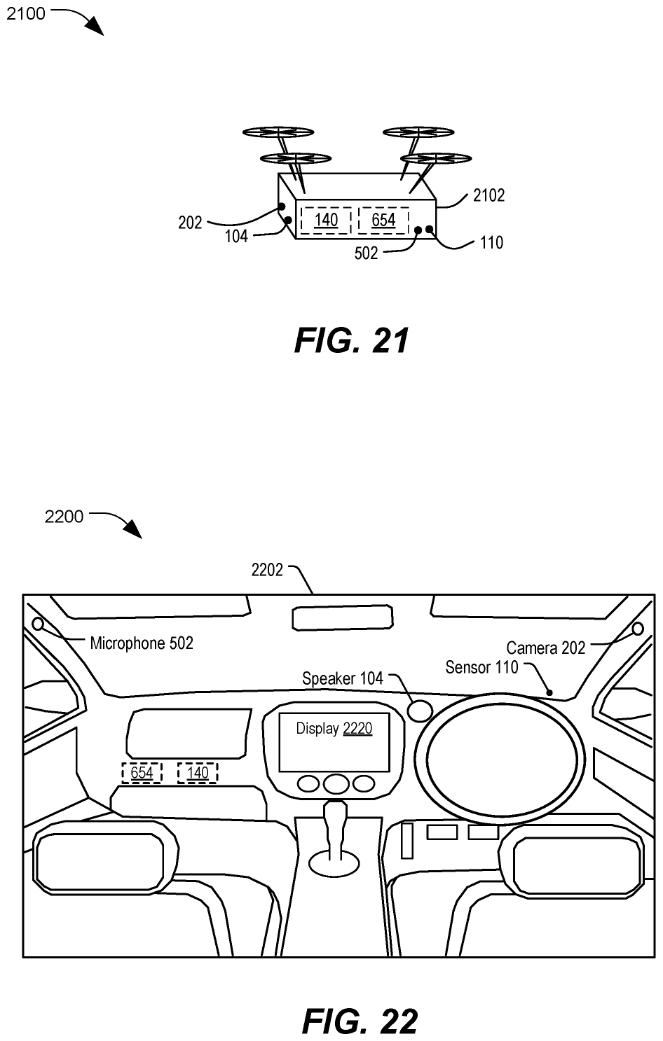

In some implementations, the device 102 corresponds to or is included in one of various types of devices. In an illustrative example, the one or more processors 190 are integrated in a headset device, such as described further with reference to FIG. 15. In other examples, the one or more processors 190 are integrated in at least one of a mobile phone or a tablet computer device, as described with reference to FIG. 14, earbuds, as described with reference to FIG. 16, a wearable electronic device, as described with reference to FIG. 17, extended reality glasses, as described with reference to FIG. 18, a voice-controlled speaker system, as described with reference to FIG. 19, or a virtual reality, mixed reality, or augmented reality headset, as described with reference to FIG. 20. In another illustrative example, the one or more processors 190 are integrated into a vehicle, such as described further with reference to FIG. 21 and FIG. 22.

During operation, the audio analyzer 140 receives, from the sensor 110, the biophysical sensor data 112 indicative of a biophysical rhythm 154 (e.g., a detected biophysical rhythm) of a person 101. In some examples, the sensor 110 includes a heart rate monitor and the biophysical sensor data 112 corresponds to a heartbeat of the person 101. In some examples, the sensor 110 includes a pedometer (e.g., in a mobile device or a wearable device) carried by the person 101, and the biophysical sensor data 112 indicates a gait cadence of the person 101. In a particular example, the sensor 110 is attached to (or integrated in) exercise equipment (e.g., a bicycle, a rowing machine, etc.), and the biophysical sensor data 112 indicates a cadence associated with the exercise equipment. In some examples, the sensor 110 includes a camera, and the biophysical sensor data 112 includes images (e.g., a video or multiple still images) of the person 101 that can be processed to estimate the biophysical rhythm 154 of the person 101.

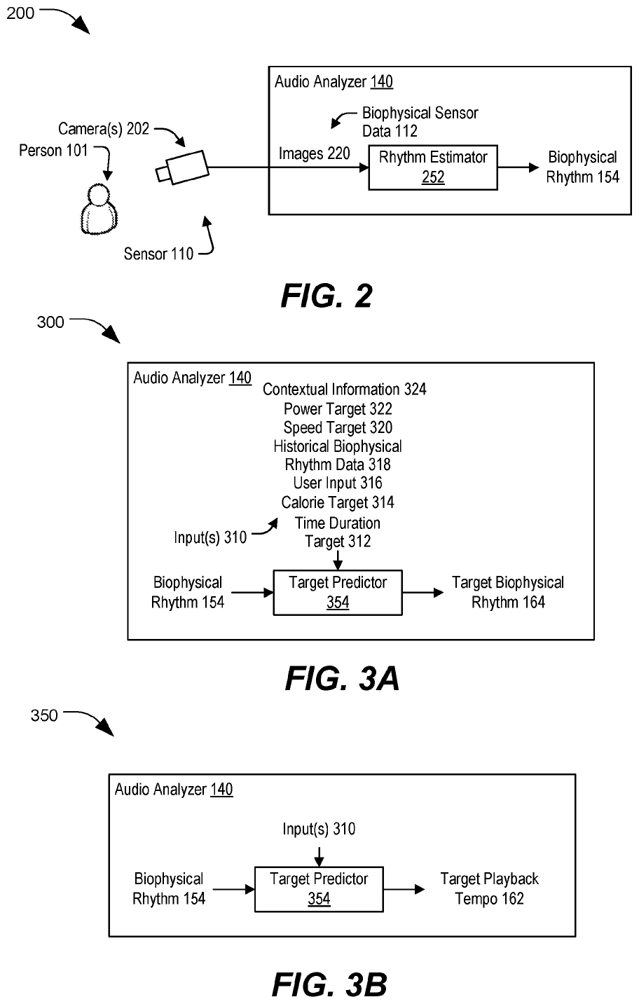

The audio analyzer 140 determines the biophysical rhythm 154 based on the biophysical sensor data 112. In some implementations, the biophysical sensor data 112 directly indicates the biophysical rhythm 154 (e.g., heartbeats per minute, steps per minute, etc.). In other implementations, the audio analyzer 140 processes the biophysical sensor data 112 (e.g., images) to determine the biophysical rhythm 154, as further described with reference to FIG. 2.

In a particular implementation, the audio analyzer 140 estimates a target biophysical rhythm 164 (e.g., 130 steps per minute) of the person 101 based on the biophysical sensor data 112, the biophysical rhythm 154, one or more other inputs, or a combination thereof, as further described with reference to FIG. 3A. In this implementation, the audio analyzer 140 determines the target playback tempo 162 (e.g., 130 BPM) based on the target biophysical rhythm 164 (e.g., 130 steps per minute). In some examples, the target biophysical rhythm 164 is the same as the biophysical rhythm 154. In other examples, the target biophysical rhythm 164 can be different from the biophysical rhythm 154.

In a particular implementation, the audio analyzer 140 estimates the target playback tempo 162 (e.g., 130 BPM) of the person 101 based on the biophysical sensor data 112, the biophysical rhythm 154, the one or more inputs 310, or a combination thereof (e.g., without an intermediate determination of the target biophysical rhythm 164), as described with reference to FIG. 3B.

In some examples, the target playback tempo 162 corresponds to a function (e.g., a linear function) applied to the target biophysical rhythm 164. In a particular aspect, when there is a 1-to-1 correspondence, the target playback tempo 162 (e.g., 130 BPM) includes one beat per step of the target biophysical rhythm 164 (e.g., 130 steps per minute). In an illustrative example, if the person 101 is listening to the audio signal 128 while walking with a gait cadence matching the target biophysical rhythm 164, each foot comes in contact with the ground in alignment with a beat of the audio. In another aspect, the target playback tempo 162 includes one beat for two steps of the target biophysical rhythm 164. In an illustrative example, if the person 101 is listening to the audio signal 128 while walking with a gait cadence matching the target biophysical rhythm 164, one of the right foot or left foot comes in contact with the ground in alignment with each beat of the audio. The function applied to the target biophysical rhythm 164 to determine the target playback tempo 162 can be based on default data, a configuration setting, a user input, or a combination thereof.

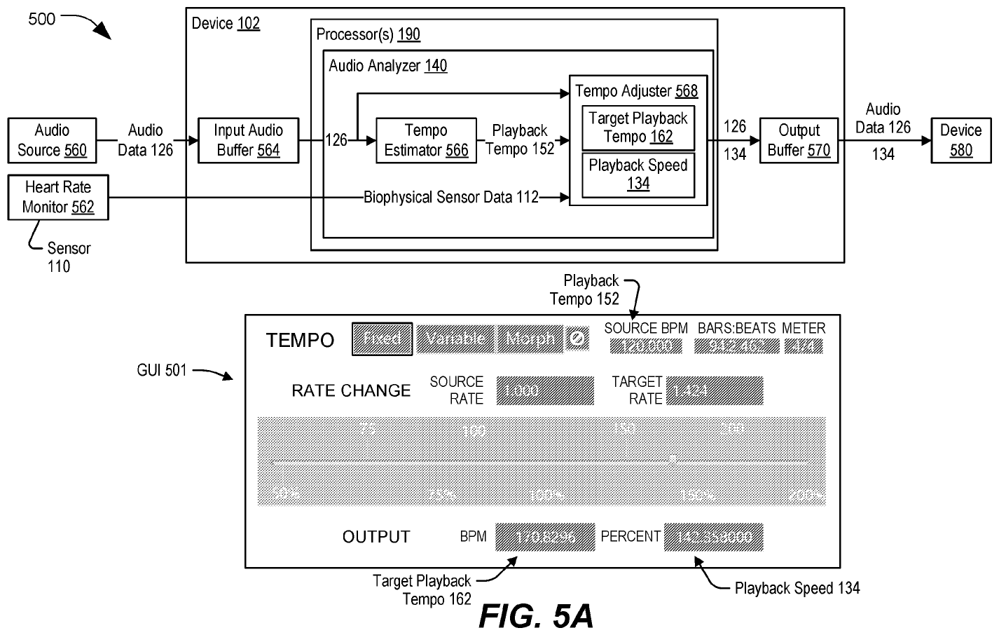

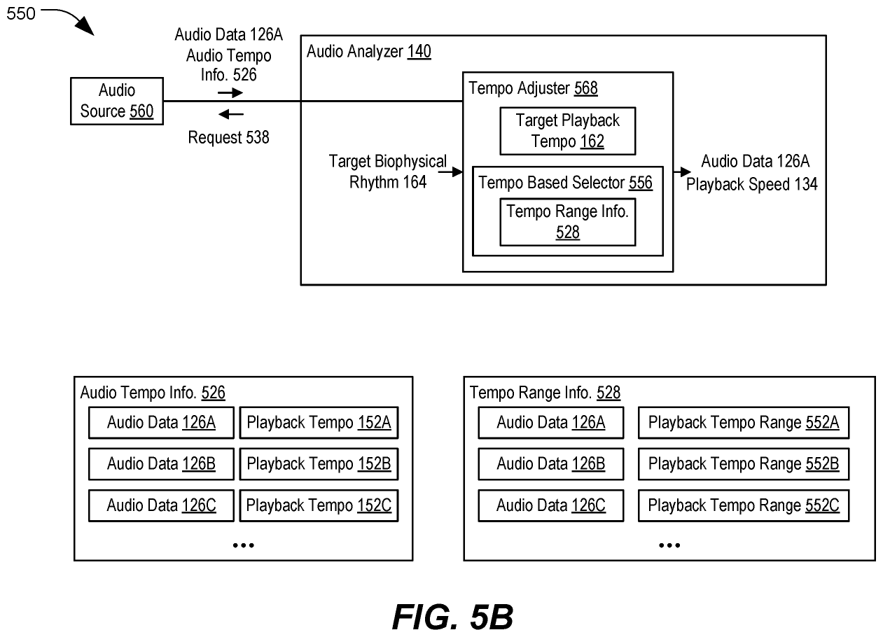

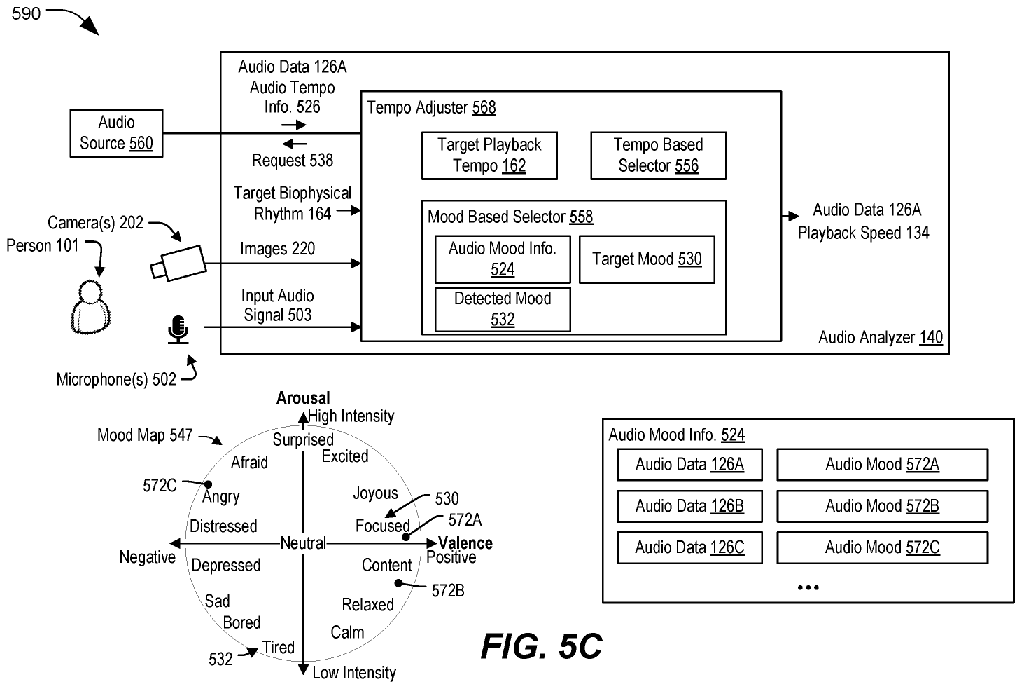

In a particular aspect, the audio data 126 has a playback tempo 152 at a particular playback speed (e.g., a default speed, an original speed, a recording speed, etc.). In some examples, the audio analyzer 140 processes the audio data 126 to determine the playback tempo 152 (e.g., 120 BPM), as described with reference to FIG. 5A. In some examples, the audio analyzer 140 obtains audio tempo information indicating that the audio data 126 has the playback tempo 152, as described with reference to FIG. 5B.

In a particular aspect, the audio analyzer 140 selects the audio data 126 based at least in part on a comparison of the playback tempo 152 and the target playback tempo 162, as further described with reference to FIGS. 5B and 5C. For example, the audio data 126 can be adjusted within particular thresholds (e.g., within −10% and +10%) of the playback tempo 152 without introducing artifacts that adversely impact the listening experience. The audio analyzer 140 selects the audio data 126 based at least in part on determining that the target playback tempo 162 is within a difference threshold (e.g., greater than or equal to 90% and less than or equal to 110%) of the playback tempo 152.

The audio analyzer 140 obtains the audio data 126 (e.g., the selected audio data) for adjustment. In some examples, the audio analyzer 140 obtains the audio data 126 from a storage device, a network device, a streaming service, or a combination thereof.

In some implementations, the audio analyzer 140 determines multiple target playback tempos 162 based on the biophysical sensor data 112, the biophysical rhythm 154, one or more inputs, or a combination thereof. For example, the audio analyzer 140 determines a first target playback tempo 162 based on the biophysical rhythm 154 and determines a second target playback tempo 162 based on the target biophysical rhythm 164 and adjusts the playback speed 134 to transition from the first target playback tempo 162 to the second target playback tempo 162. In a particular aspect, the playback speed 134 is to be adjusted multiple times to transition from the first target playback tempo 162, via one or more intermediate target playback tempos, to the second target playback tempo 162. A technical advantage of adjusting the playback speed 134 multiple times can include avoiding a sudden jump from the first target playback tempo 162 to the second target playback tempo 162. In these implementations, the audio analyzer 140 selects the audio data 126 based at least in part on determining that each of the first target playback tempo 162 and the second target playback tempo 162 is within the particular thresholds (e.g., greater than or equal to 90% and less than or equal to 110%) of the playback tempo 152.

The audio analyzer 140 determines the playback speed 134 (e.g., 130/120=108%) based on the playback tempo 152 and the target playback tempo 162 (e.g., the playback speed 134=the target playback tempo 162/the playback tempo 152). The audio analyzer 140 adjusts (e.g., sets) a playback speed of the audio data 126 to the playback speed 134 so that the audio data 126 has the target playback tempo 162 that matches the target biophysical rhythm 164. The audio analyzer 140 initiates playback, via the one or more speakers 104, of an audio signal 128 corresponding to the audio data 126 having the playback speed 134 (e.g., 108%). For example, the one or more speakers 104, during playback of the audio signal 128, output audio corresponding to the audio signal 128.

The system 100 thus automatically adjusts playback speed of the audio data 126, based at least in part on the biophysical sensor data 112. A technical advantage of the automatic playback speed adjustment can include the audio data 126 having the target playback tempo 162 that matches the biophysical rhythm 154 indicated by the biophysical sensor data 112. Listening to the audio signal 128 corresponding to the audio data 126 having the target playback tempo 162 can aid the person 101 in reaching and maintaining the target biophysical rhythm 164.

Although the sensor 110 and the one or more speakers 104 are illustrated as being coupled to the device 102, in other implementations one or more of the sensor 110 and the one or more speakers 104 may be integrated in the device 102. Although a single sensor 110 is illustrated, in other implementations multiple sensors configured to generate biophysical sensor data 112 may be included.

Referring to FIG. 2, a diagram 200 is shown of an illustrative aspect of operations associated with a rhythm estimator 252. The rhythm estimator 252 is coupled to the sensor 110. In some implementations, the sensor 110 includes one or more cameras 202.

The one or more cameras 202 are configured to capture images 220 of the person 101. In some examples, the images 220 correspond to image frames of a video. In other examples, the images 220 correspond to still images (e.g., from a photo burst). The rhythm estimator 252 receives the images 220 from the one or more cameras 202 and processes the images 220 to estimate the biophysical rhythm 154. For example, the rhythm estimator 252 estimates a gait cadence, a swim cadence, or a cycling cadence of the person 101 based on the images 220 indicating that the person 101 is walking (or running), swimming, or cycling, respectively. In some examples, the rhythm estimator 252 estimates a heartbeat, a respiration rate, or both, based on the images 220. To illustrate, the rhythm estimator 252 can estimate the heartbeat based on color changes in the skin that indicate a pulse rate. In a particular aspect, the rhythm estimator 252 determines the biophysical rhythm 154 based on timestamps of the images 220 and changes in position of the person 101 detected in the images 220. The rhythm estimator 252 outputs the gait cadence, the swim cadence, the cycling cadence, the heartbeat, the respiration rate, or a combination thereof, as the biophysical rhythm 154.

Optionally, in some implementations, the sensor 110 generates the biophysical sensor data 112 that directly indicates the biophysical rhythm 154. For example, the sensor 110 includes a heart rate monitor that outputs the biophysical sensor data 112 indicating a heart rate (e.g., heart beats per minute) as the biophysical rhythm 154. In another example, the sensor 110 includes a pedometer that outputs the biophysical sensor data 112 indicating a gait cadence (e.g., steps per minute) as the biophysical rhythm 154. In these implementations, the rhythm estimator 252 outputs the biophysical rhythm 154 indicated by the biophysical sensor data 112.

Referring to FIG. 3A, a diagram 300 is shown of an illustrative aspect of operations associated with a target predictor 354 included in the audio analyzer 140. The target predictor 354 is configured to predict the target biophysical rhythm 164.

In a particular implementation, the target predictor 354 determines the target biophysical rhythm 164 based on the biophysical rhythm 154. In some examples, the target predictor 354 determines the target biophysical rhythm 164 based on one or more inputs 310 in addition to the biophysical rhythm 154. The one or more inputs 310 can include a time duration target 312, a calorie target 314, a user input 316, historical biophysical rhythm data 318, a speed target 320, a power target 322, contextual information 324, or a combination thereof. For example, the target predictor 354, in response to determining that the calorie target 314 can be reached during the time duration target 312 at a particular biophysical rhythm, outputs the particular biophysical rhythm as the target biophysical rhythm 164.

In some implementations, the time duration target 312 indicates that a session (e.g., an exercise session) is to last a particular duration (e.g., 30 minutes). In some implementations, the time duration target 312 indicates that a particular duration of the session is to be greater than or equal to a first duration threshold (e.g., 20 minutes) and less than or equal to a second duration threshold (e.g., 40 minutes). In some implementations, the calorie target 314 indicates a particular calorie count (e.g., 150 calories burned) is to be achieved during the session. In some implementations, the calorie target 314 indicates that a particular calorie count achieved during the session is to be greater than or equal to a first calorie threshold and less than or equal to a second calorie threshold.

In some implementations, the speed target 320 indicates that a particular speed (e.g., 3 miles an hour) is to be maintained during a majority of a session (e.g., an exercise session). In some implementations, the speed target 320 indicates that a particular speed during a majority of the session is to be greater than or equal to a first speed threshold (e.g., 2 miles an hour) and less than or equal to a second speed threshold (e.g., 4 miles an hour). In some implementations, the power target 322 indicates that a particular power level (e.g., 130 watts per hour) is to be achieved during the session. In a cycling example, a power level is based on a gear and rotations per minute (RPM). A higher gear, a higher RPM, or both, correspond to a higher power level during the session. In some implementations, the power target 322 indicates that a particular power level achieved during the session is to be greater than or equal to a first power level threshold (e.g., 120 watts per hour) and less than or equal to a second power level threshold (e.g., 140 watts per hour).

The contextual information 324 can indicate external conditions, such as terrain (e.g., uphill, downhill, etc.), environmental conditions (e.g., precipitation, temperature, etc.), surface type (e.g., gravel, asphalt, dirt, etc.), traffic, or a combination thereof. In some aspects, the contextual information 324 can include global positioning system (GPS) data, weather forecast data, traffic data, sensor data, or a combination thereof.

In a particular aspect, the target predictor 354 includes a trained model (e.g., a graph convolutional network (GCN)) that processes the target biophysical rhythm 164, the one or more inputs 310, or a combination thereof, to predict the target biophysical rhythm 164, as further described with reference to FIG. 4A.

In some implementations, the target predictor 354 determines the target biophysical rhythm 164 based on the biophysical sensor data 112, the one or more inputs 310, or a combination thereof (e.g., without an intermediate operation of the audio analyzer 140 determining the biophysical rhythm 154). For example, the target predictor 354 determines the target biophysical rhythm 164 independently of determining the biophysical rhythm 154. To illustrate, the target predictor 354 determines the target biophysical rhythm 164 based on the biophysical sensor data 112, that is indicative of the biophysical rhythm 154, without explicitly determining the biophysical rhythm 154.

Referring to FIG. 3B, a diagram 350 is shown of an illustrative aspect of operations associated with a target predictor 354 included in the audio analyzer 140. The target predictor 354 is configured to predict the target playback tempo 162 (e.g., without an intermediate operation of determining the target biophysical rhythm 164).

In a particular implementation, the target predictor 354 determines the target playback tempo 162 based on the biophysical rhythm 154. In some examples, the target predictor 354 determines the target playback tempo 162 based the one or more inputs 310 in addition to the biophysical rhythm 154. For example, the target predictor 354 uses a trained model (e.g., a GCN) to process the biophysical rhythm 154, the one or more inputs 310, or a combination thereof, to predict the target playback tempo 162, as further described with reference to FIG. 4B.

In some implementations, the target predictor 354 determines the target playback tempo 162 based on the biophysical sensor data 112, the one or more inputs 310, or a combination thereof (e.g., without an intermediate operation of determining the biophysical rhythm 154). For example, the target predictor 354 determines the target playback tempo 162 independently of determining the biophysical rhythm 154. To illustrate, the target predictor 354 determines the target playback tempo 162 based on the biophysical sensor data 112, that is indicative of the biophysical rhythm 154, without explicitly determining the biophysical rhythm 154.

Referring to FIG. 4A, a diagram of an illustrative aspect of operations associated with a graph convolutional network (GCN) 400 is shown. In a particular aspect, the target predictor 354 of FIG. 3A includes the GCN 400.

The GCN 400 corresponds to a set of equations 402 associated with a mixed integer program (MIP) that predicts the target biophysical rhythm 164 given a set of linear constraints and integer variables. For example, the set of equations 402 are based on variables (e.g., b₁, . . . , bₙ, where n is an integer greater than 0) and constraints (e.g., b1, . . . , bₘ, where m is an integer greater than 0 and m can be less than, equal to, or greater than n). The GCN 400 includes variable nodes 420 and constraint nodes 430.

Each of the variable nodes 420 corresponds to a variable of the set of equations 402, and each of the constraint nodes 430 corresponds to a constraint of the set of equations 402.

In an example, a variable can include the biophysical rhythm 154, the target biophysical rhythm 164, the user input 316, the historical biophysical rhythm data 318, the contextual information 324, one or more additional variables, or a combination thereof. In an example, a constraint can include the time duration target 312, the calorie target 314, the speed target 320, the power target 322, one or more additional constraints, or a combination thereof. The coefficients of the set of equations 402 correspond to features of the nodes and edges of the GCN 400.

The GCN 400 is provided as a non-limiting illustrative implementation of the target predictor 354 of FIG. 3A. In other implementations, the target predictor 354 can include other types of neural networks, such as Message Passing Neural Networks (MPNNs), Graph Attention Networks (GATs), or Deep Neural Networks (DNNs). In some implementations, the target predictor 354 includes a neural network that does not rely on graph topologies or any convolutional layers. For example, Recurrent Neural Network (RNN) layers are suitable alternatives to convolutional layers, especially for time-series prediction that track changes over time. An example of such recurrent layers are LSTM (Long Short Term Memory) layers or GRUs (Gated Recurrent Units).

Referring to FIG. 4B, a diagram of an illustrative aspect of operations associated with a GCN 450 is shown. In a particular aspect, the target predictor 354 of FIG. 3B includes the GCN 450.

The GCN 450 corresponds to a set of equations 402 associated with a MIP that predicts the target playback tempo 162 given a set of linear constraints and integer variables. For example, at least one of the variable nodes 420 corresponds to the target playback tempo 162.

The GCN 450 is provided as a non-limiting illustrative implementation of the target predictor 354 of FIG. 3B. In other implementations, the target predictor 354 can include other types of neural networks, such as Recurrent Neural Networks, Message Passing Neural Networks, Graph Attention Networks, Deep Neural Networks, etc.

Referring to FIG. 5A, a diagram is shown of an illustrative aspect of a system 500 that is operable to adjust audio playback speed based on an estimated playback tempo. In a particular aspect, the system 100 of FIG. 1 includes one or more components of the system 500.

The audio analyzer 140 is coupled to an input audio buffer 564, an output buffer 570, or both. In a particular aspect, the input audio buffer 564 is configured to be coupled to an audio source 560. The audio source 560 can include a storage device, a streaming service, a network device, another type of device, or a combination thereof.

In a particular aspect, the output buffer 570 is configured to be coupled to a device 580. The device 580 can include a user device, a network device, a playback device, a headset, earbuds, a speaker, or a combination thereof.

The audio analyzer 140 includes a tempo adjuster 568 coupled to the input audio buffer 564, the sensor 110, or both. As an example, the sensor 110 includes a heart rate monitor 562. In some implementations, the audio analyzer 140 includes a tempo estimator 566 coupled to the input audio buffer 564 and to the tempo adjuster 568.

The device 102 receives the audio data 126 from the audio source 560 and stores the audio data 126 in the input audio buffer 564. In a particular aspect, the audio data 126 corresponds to a stream of audio frames. The tempo estimator 566 uses various audio analysis techniques to determine the playback tempo 152 (e.g., 120 BPM) of the audio data 126. The tempo adjuster 568 receives the biophysical sensor data 112 from the sensor 110 (e.g., the heart rate monitor 562). The tempo adjuster 568 determines the target playback tempo 162 based at least in part on the biophysical sensor data 112, the biophysical rhythm 154, the one or more inputs 310, or a combination thereof, as described with reference to FIGS. 3A-3B.

The tempo adjuster 568 determines the playback speed 134 (e.g., 142.358%) based on a comparison of the playback tempo 152 and the target playback tempo 162 (e.g., the playback speed 134=the target playback tempo 162/the playback tempo 152). The tempo adjuster 568 adjusts a playback speed of the audio data 126 to the playback speed 134 so that the audio data 126 at the playback speed 134 has the target playback tempo 162. The tempo adjuster 568 provides the audio data 126, the playback speed 134, or both, to the output buffer 570. In a particular implementation, the device 102 sends the audio data 126, the playback speed 134, or both, from the output buffer 570 to the device 580. In an alternative implementation, the device 102 sends the audio signal 128 of FIG. 1 corresponding to the audio data 126 having the playback speed 134 from the output buffer 570 to the device 580 (e.g., a speaker).

In a particular implementation, the audio analyzer 140 generates a graphical user interface (GUI) 501 indicating the playback tempo 152, the target playback tempo 162, the playback speed 134, or a combination thereof. In some examples, a user (e.g., the person 101 or another user) can use an input control (e.g., a slider) of the GUI 501 to override the automatic playback speed adjustment.

Optionally, in some implementations in which the audio analyzer 140 does not include the tempo estimator 566, the audio analyzer 140 can determine the playback tempo 152 based on received tempo information, as further described with reference to FIG. 5B. A technical advantage of using the tempo estimator 566 to determine the playback tempo 152 is independence from having to obtain the tempo information. A technical advantage of determining the playback tempo 152 based on the received tempo information can include fewer computing cycles, less time, or both, to determine the playback tempo 152. In some examples, the audio analyzer 140 selectively uses the tempo estimator 566 to determine the playback tempo 152 in response to determining that tempo information indicating the playback tempo 152 of the audio data 126 is unavailable.

Referring to FIG. 5B, a diagram is shown of an illustrative aspect of a system 550 that is operable to adjust audio playback speed based on received tempo information. In a particular aspect, the system 100 of FIG. 1 includes one or more components of the system 550.

The tempo adjuster 568 receives audio tempo information 526 from the audio source 560 or from another device. The audio tempo information 526 indicates a mapping between sets of audio data 126 and playback tempos 152. For example, the audio tempo information 526 indicates that audio data 126A, audio data 126B, and audio data 126C correspond to a playback tempo 152A, a playback tempo 152B, and a playback tempo 152C, respectively. The audio tempo information 526 including three mappings is described as an illustrative example, in other examples the audio tempo information 526 can include fewer than three or more than three mappings.

The tempo adjuster 568 is configured to adjust a playback speed of audio data 126 based on the target playback tempo 162 and the corresponding playback tempo 152 indicated by the audio tempo information 526. For example, the tempo adjuster 568 determines the playback speed 134 based on the target playback tempo 162 and the playback tempo 152, as described with reference to FIG. 1.

In some implementations, the tempo adjuster 568 includes a tempo based selector 556 that is configured to select one of multiple sets of audio data 126 based on a corresponding playback tempo 152 and the target playback tempo 162. The tempo based selector 556 generates tempo range information 528 based on the audio tempo information 526. For example, the tempo based selector 556 determines a playback tempo range 552 of audio data 126 based on a playback tempo 152 of the audio data 126 and a difference threshold (e.g., +/−10%). Playback speed adjustments that are beyond the difference threshold (e.g., slower than 90% or faster than 110%) are likely to introduce artifacts that are considered intolerable. A technical advantage of playback speed adjustments that are within the difference threshold is that such playback speed adjustments are likely to introduce tolerable artifacts, if any, in the audio data 126.

The tempo based selector 556 adds a mapping between the audio data 126 and the playback tempo range 552 (e.g., 90% of the playback tempo 152 to 110% of the playback tempo 152) to the tempo range information 528. In an illustrative example, the tempo based selector 556 updates the tempo range information 528 to indicate that the audio data 126A, the audio data 126B, and the audio data 126C are associated with a playback tempo range 552A (e.g., 90% of the playback tempo 152A to 110% of the playback tempo 152A), a playback tempo range 552B (e.g., 90% of the playback tempo 152B to 110% of the playback tempo 152B), and a playback tempo range 552C (e.g., 90% of the playback tempo 152C to 110% of the playback tempo 152C), respectively. The tempo range information 528 including three mappings is described as an illustrative example, in other examples the tempo range information 528 can include fewer than three or more than three mappings.

The tempo based selector 556 selects the audio data 126A based at least in part on determining that the audio data 126A satisfies the target playback tempo 162. For example, the audio data 126A satisfies the target playback tempo 162 if the target playback tempo 162 is within the playback tempo range 552A (e.g., greater than or equal to 90% of the playback tempo 152A and less than or equal to 110% of the playback tempo 152A) of the audio data 126A.

In some implementations, if multiple sets of audio data 126 satisfy the target playback tempo 162, the tempo based selector 556 selects one of the multiple sets having a playback tempo 152 that is closest to the target playback tempo 162. For example, the tempo based selector 556, in response to determining that the target playback tempo 162 is within the playback tempo range 552A and within the playback tempo range 552B, selects the audio data 126A if the playback tempo 152A is closer to the target playback tempo 162 than the playback tempo 152B is to the target playback tempo 162. To illustrate, the tempo based selector 556 selects the audio data 126A if a first difference between the playback tempo 152A and the target playback tempo 162 is less than or equal to a second difference between the playback tempo 152B and the target playback tempo 162. A technical advantage of selecting the audio data 126A is that the lower first difference corresponds to a smaller playback speed adjustment, thereby introducing fewer artifacts in the audio signal 128 of FIG. 1.

In some examples, the audio analyzer 140 is to transition playback between multiple target playback tempos 162. In these examples, the audio analyzer 140 selects audio data 126 that satisfies more of the multiple target playback tempos 162. In an example, the audio analyzer 140 is to transition playback between a first target playback tempo 162 and a second target playback tempo 162. In an illustrative example, the audio analyzer 140 determines that the audio data 126A satisfies both the first target playback tempo 162 and the second target playback tempo 162. The audio analyzer 140 also determines that the audio data 126B does not satisfy the first target playback tempo 162 and satisfies the second target playback tempo 162. Additionally, the audio data 126C does not satisfy either of the first target playback tempo 162 or the second target playback tempo 162. In this example, the tempo based selector 556 selects the audio data 126A because the audio data 126A satisfies more of the multiple target playback tempos 162 (e.g., both of the first target playback tempo 162 and the second target playback tempo 162) as compared to each of the audio data 126B and the audio data 126C. A technical advantage of selecting the audio data 126A is that the audio data 126A satisfying more of the multiple target playback tempos 162 can correspond to fewer switches between sets of the audio data 126 as the audio analyzer 140 transitions playback between multiple target playback tempos 162.

The audio analyzer 140 obtains the selected audio data (e.g., the audio data 126A). In a particular aspect, the audio analyzer 140 sends a request 538 to the audio source 560. The request 538 indicates the selected audio data (e.g., the audio data 126A). The audio analyzer 140, responsive to sending the request 538, receives the audio data 126A from the audio source 560.

Referring to FIG. 5C, a diagram is shown of an illustrative aspect of a system 590 that is operable to adjust audio playback speed based on a detected mood. In a particular aspect, the system 100 of FIG. 1 includes one or more components of the system 590.

The tempo adjuster 568 includes a mood based selector 558 that is configured to select audio data based at least in part on a detected mood 532, a target mood 530, or both. In a particular aspect, the tempo adjuster 568 is coupled to one or more microphones 502.

The mood based selector 558 obtains audio mood information 524. In a particular implementation, the mood based selector 558 obtains at least a portion of the audio mood information 524 from the audio source 560, another information source, or both. In a particular implementation, the mood based selector 558 generates at least a portion of the audio mood information 524. For example, the mood based selector 558 uses various audio mood analysis techniques to determine that the audio data 126A is associated with an audio mood 572A (e.g., sad, happy, angry, energetic, mellow, or a combination thereof). In a particular aspect, the mood based selector 558 determines the audio mood 572A based on the playback tempo 152A of the audio data 126A, a music genre associated with the audio data 126A, or both.

The audio mood information 524 indicates mappings between sets of the audio data 126 and audio moods 572. For example, the audio mood information 524 indicates that the audio data 126A, the audio data 126B, and the audio data 126C have the audio mood 572A, the audio mood 572B, and the audio mood 572C, respectively. The audio mood information 524 including three mappings is described as an illustrative example, in other examples the audio mood information 524 can include fewer than three or more than three mappings.

In a particular aspect, a particular mood is associated with a particular value on a mood map 547. In some examples, a horizontal value (e.g., an x-coordinate) of the particular value indicates valance of the particular mood, and a vertical value (e.g., a y-coordinate) of the particular value indicates intensity of the particular mood. A distance (e.g., a Cartesian distance) between a pair of moods indicates a similarity between the moods. For example, the mood map 547 indicates a first distance (e.g., a first Cartesian distance) between first coordinates corresponding to the audio mood 572A and second coordinates corresponding to the audio mood 572B and a second distance (e.g., a second Cartesian distance) between the first coordinates corresponding to the audio mood 572A and third coordinates corresponding to the audio mood 572C. The first distance is less than the second distance indicating that the audio mood 572A has greater similarity with the audio mood 572B than with the audio mood 572C. The mood map 547 is illustrated as a two-dimensional space as a non-limiting example. In other examples, the mood map 547 can be a multi-dimensional space.

The detected mood 532 includes a user mood, a scene mood, or both. In a particular aspect, the mood based selector 558 determines the detected mood 532 based on images 220 received from the one or more cameras 202, an input audio signal 503 received from the one or more microphones 502, or a combination thereof. For example, the images 220 include at least one image of the person 101 and the mood based selector 558 uses various user image analysis techniques to process at least one image of the person 101 to estimate the user mood. In another example, the mood based selector 558 uses various image mood analysis techniques to process the images 220 to estimate the scene mood. To illustrate, the mood based selector 558, in response to detecting that the images 220 indicate a running track, estimates that the scene mood is energetic.

In a particular aspect, the mood based selector 558 determines the target mood 530 based on the detected mood 532, a user input, user calendar data, default data, a configuration setting, or a combination thereof. For example, the mood based selector 558, in response to determining that the user calendar data indicates work hours, estimates that the target mood 530 corresponds to a focused mood. In another example, the mood based selector 558, in response to determining that a detected valence of the detected mood 532 is negative, generates the target mood 530 with a target valence that is positive relative to the detected valence. To illustrate, the target valence corresponds to a sum of the detected valence and a predetermined target valence difference, where the predetermined target valence difference is based on default data, a configuration setting, a user input, or a combination thereof. As another example, the mood based selector 558 sets the target mood 530 to be the same as the detected mood 532 (e.g., the user mood, the scene mood, or both).

The mood based selector 558 selects the audio data 126A based at least in part on the target mood 530. For example, the mood based selector 558 selects the audio data 126A based on determining that the audio data 126A matches the target mood 530. The audio data 126A matches the target mood 530 if the audio mood 572A of the audio data 126A matches the target mood 530. In an example, the audio mood 572A matches the target mood 530 if a distance (e.g., Cartesian distance) between the first coordinates of the audio mood 572A is within a distance threshold of target coordinates of the target mood 530.

In a particular example, the mood based selector 558 selects the audio data 126A based on determining that the target mood 530 has greater similarity with the audio mood 572A than with each of the audio mood 572B and the audio mood 572C.

In a particular implementation, the tempo based selector 556 selects a first subset of the sets of audio data 126 based on the target playback tempo 162, as described with reference to FIG. 5B. If the first subset includes multiple sets of audio data 126, the mood based selector 558 selects the audio data 126A from the first subset based on the target mood 530. In an alternative implementation, the mood based selector 558 selects a first subset of the sets of audio data 126 based on the target mood 530. If the first subset includes multiple sets of audio data 126, the tempo based selector 556 selects the audio data 126A from the first subset based on the target playback tempo 162, as described with reference to FIG. 5B. In some implementations, the audio data 126A can be selected based at least in part on one or more other criteria, such as a user preference, a user playlist, an age restriction, an audio service membership, a cost associated with providing the audio data 126A to the person 101, or a combination thereof.

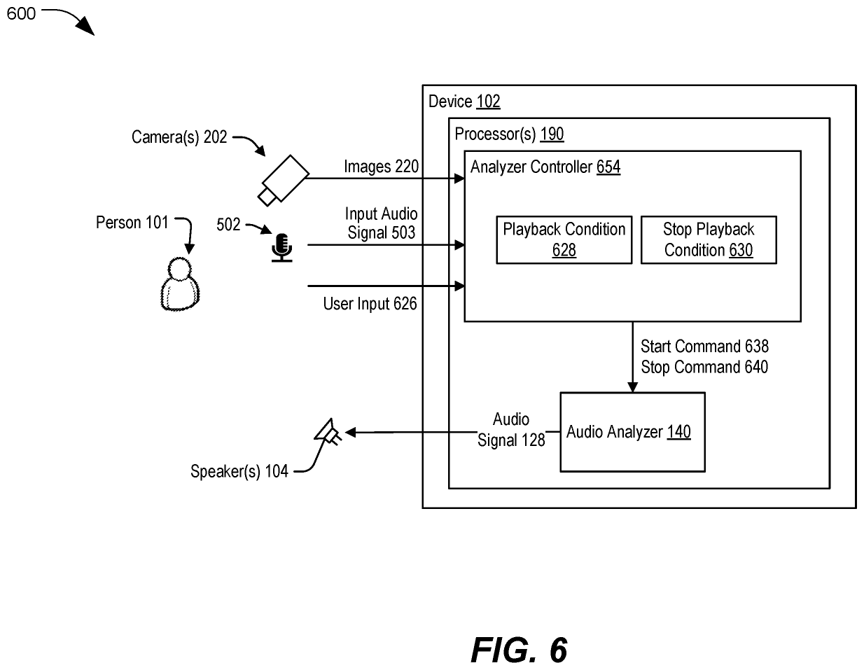

Referring to FIG. 6, a diagram is shown of an illustrative implementation of a system 600 that is operable to adjust audio playback speed responsive to detection of a playback condition. In a particular aspect, the system 100 of FIG. 1 includes one or more components of the system 600. The one or more processors 190 include an analyzer controller 654 coupled to the audio analyzer 140.

The analyzer controller 654 is configured to, in response to detecting a playback condition 628, send a start command 638 to the audio analyzer 140 to activate the audio analyzer 140 to adjust audio playback speed, initiate audio playback, or both. The analyzer controller 654 is configured to, in response to detecting a stop playback condition 630, send a stop command 640 to the audio analyzer 140 to deactivate the audio analyzer 140 to refrain from adjusting audio playback speed, discontinue audio playback, or both.

In a particular aspect, the playback condition 628, the stop playback condition 630, or both, are based on default data, a configuration setting, a user input, or a combination thereof. For example, the playback condition 628 can include the person 101 starting an exercise session, and the stop playback condition 630 can include the person 101 ending the exercise session.

When the audio analyzer 140 is deactivated, the analyzer controller 654 processes the images 220, the input audio signal 503, a user input 626, or a combination thereof, to determine whether the playback condition 628 is detected. In a particular aspect, the analyzer controller 654 checks for the playback condition 628 at various time intervals, responsive to user input activating the analyzer controller 654, or a combination thereof. Alternatively, when the audio analyzer 140 is activated, the analyzer controller 654 processes the images 220, the input audio signal 503, a user input 626, or a combination thereof, to determine whether the stop playback condition 630 is detected. In a particular aspect, the analyzer controller 654 checks for the stop playback condition 630 at various time intervals, responsive to user input activating the analyzer controller 654, or a combination thereof.

The analyzer controller 654, in response to detecting the playback condition 628, sends the start command 638 to the audio analyzer 140. The audio analyzer 140, in response to receiving the start command 638, initiates playback of the audio signal 128 via the one or more speakers 104. In a first example, the one or more processors 190, prior to the audio analyzer 140 receiving the start command 638, are not outputting an audio signal corresponding to any audio data 126. In a particular aspect, the audio analyzer 140 selects the audio data 126A based on the playback tempo 152A, the audio mood 572A, or both, as described with reference to FIGS. 5B-5C. In a particular aspect, the audio analyzer 140 selects the audio data 126A based on the user input 626 indicating a user selection of the audio data 126A. The one or more processors 190 initiate output of the audio signal 128 corresponding to the audio data 126A having the playback speed 134, as described with reference to FIG. 1.

In a second example, the one or more processors 190 are, prior to the audio analyzer 140 receiving the start command 638, outputting an audio signal corresponding to the audio data 126A at a playback speed associated with the playback tempo 152A. The audio analyzer 140, in response to receiving the start command 638, determines whether the audio data 126A satisfies a selection criterion. For example, the selection criterion is satisfied if the audio data 126A satisfies the target playback tempo 162, matches the target mood 530, or both, as described with reference to FIGS. 5A-5B. In a particular aspect, the selection criterion is satisfied if the audio analyzer 140 receives the user input 626 indicating a user selection of the audio data 126A. The audio analyzer 140, in response to determining that the audio data 126A satisfies the selection criterion, outputs the audio signal 128 corresponding to the audio data 126A having the playback speed 134. Alternatively, the audio analyzer 140, in response to determining that the audio data 126A fails to satisfy the selection criterion, selects another set of audio data (e.g., the audio data 126B) that satisfies the selection criterion, discontinues playback of the audio signal corresponding to the audio data 126A and initiates playback via the one or more speakers 104 of the audio signal 128 corresponding to the audio data 126B having the playback speed 134.

The analyzer controller 654, in response to detecting the stop playback condition 630, sends the stop command 640 to the audio analyzer 140. For example, the analyzer controller 654 detects the stop playback condition 630 based on the images 220, the input audio signal 503, the user input 626, or a combination thereof, received during playback of the audio signal 128. In a particular aspect, the audio analyzer 140, in response to receiving the stop command 640 and determining that the audio signal 128 is being output, discontinues playback of the audio signal 128. In some aspects, the audio analyzer 140, responsive to receiving the stop command 640, continues playback of an audio signal corresponding to the audio data 126 without the playback speed adjustment. For example, the audio analyzer 140, in response to receiving the stop command 640, initiates playback of an audio signal corresponding to the audio data 126 at a playback speed (e.g., an original speed or a default speed) associated with the playback tempo 152.

A single device (e.g., the device 102) including the analyzer controller 654 and the audio analyzer 140 is provided as an illustrative example. In other examples, the analyzer controller 654 can be included in another device that sends the start command 638 or the stop command 640 to the device 102 that includes the audio analyzer 140.

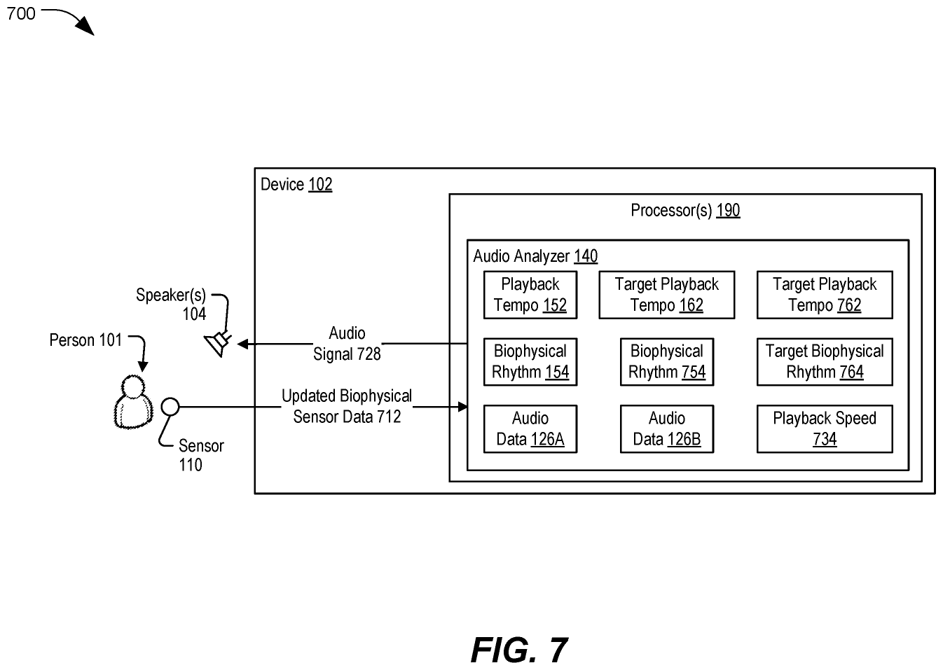

Referring to FIG. 7, a diagram of an illustrative implementation of a system 700 operable to adjust audio playback speed based on updated biophysical sensor data is shown. In a particular aspect, the system 100 of FIG. 1 includes one or more components of the system 700.

The audio analyzer 140 receives updated biophysical sensor data 712 indicating a biophysical rhythm 754 of the person 101. In an example, the audio analyzer 140, based on the updated biophysical sensor data 712, detects a change in the biophysical rhythm of the person 101 from the biophysical rhythm 154 to the biophysical rhythm 754. In a particular aspect, the audio analyzer 140 receives the updated biophysical sensor data 712 while the audio signal 128 is being output via the one or more speakers 104, where the audio signal 128 corresponds to the audio data 126A having the playback speed 134. In a particular aspect, the audio analyzer 140, in response to determining that a difference between the biophysical rhythm 754 and the biophysical rhythm 154 is greater than a rhythm change threshold, determines that an audio adjustment is to be performed.

In a particular aspect, the audio analyzer 140, in response to determining that an audio adjustment is to be performed, determines a target biophysical rhythm 764 based at least in part on the biophysical rhythm 754, as described with reference to FIG. 3A. In this aspect, the audio analyzer 140 determines a target playback tempo 762 that matches the target biophysical rhythm 764, as described with reference to FIG. 1. In an alternative aspect, the audio analyzer 140, in response to determining that an audio adjustment is to be performed, determines the target playback tempo 762 based at least in part on the biophysical rhythm 754 (e.g., without an intermediate operation of determining the target biophysical rhythm 764), as described with reference to FIG. 3B.

The audio analyzer 140 determines whether the audio data 126A satisfies a selection criterion. For example, the selection criterion is satisfied if the audio data 126A matches the target playback tempo 762, a target mood, or both, as described with reference to FIGS. 5B-5C. In a particular aspect, the selection criterion is satisfied if the audio analyzer 140 receives a user input indicating a user selection of the audio data 126A.

The audio analyzer 140, in response to determining that the audio data 126A satisfies the selection criterion, determines a playback speed 734 based on the target playback tempo 762 and the playback tempo 152A, and outputs an audio signal 728 corresponding to the audio data 126A having the playback speed 734 so that the audio data 126A has the target playback tempo 762. Alternatively, the audio analyzer 140, in response to determining that the audio data 126A fails to satisfy the selection criterion, selects another set of audio data (e.g., the audio data 126B) that satisfies the selection criterion, obtains the audio data 126B and determines the playback speed 734 based on the target playback tempo 762 and the playback tempo 152B. The audio analyzer 140 adjusts a playback speed of the audio data 126B to the playback speed 734 so that the audio data 126B has the target playback tempo 762. The audio analyzer 140 outputs the audio signal 728 corresponding to the audio data 126B having the playback speed 734.

The audio analyzer 140 can thus automatically update the audio signal output via the one or more speakers 104 based on the updated biophysical sensor data 712. For example, the audio signal can correspond to the audio data 126A or the audio data 126B having the target playback tempo 762.

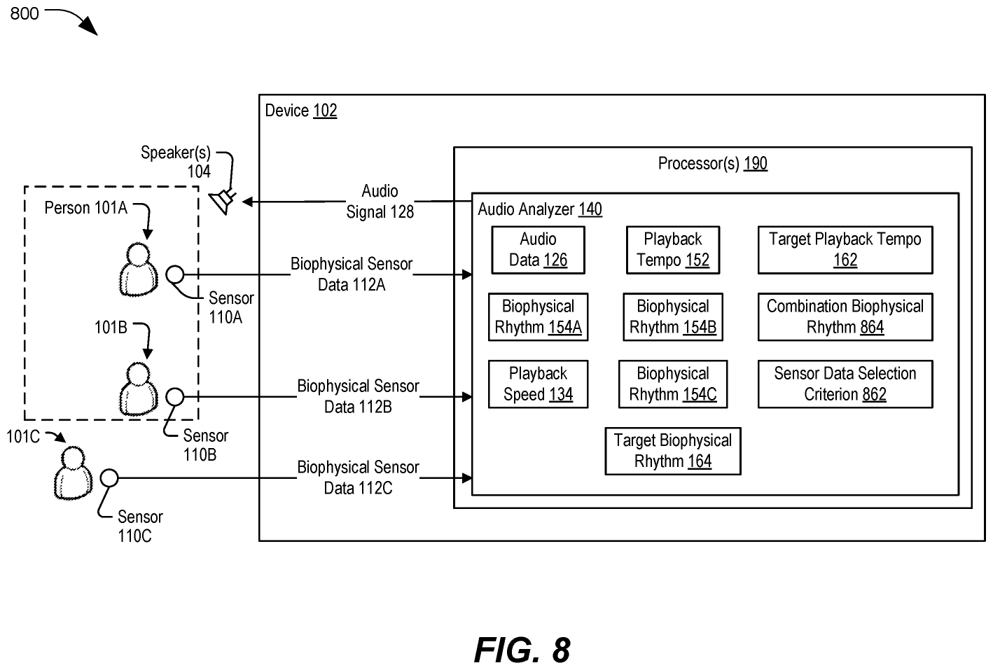

Referring to FIG. 8, a diagram is shown of an illustrative implementation of a system 800 that is operable to adjust audio playback speed based on biophysical sensor data that satisfies a sensor data selection criterion 862. In a particular aspect, the system 100 of FIG. 1 includes one or more components of the system 800.

The audio analyzer 140 receives biophysical sensor data indicative of detected biophysical rhythms of one or more persons. For example, the audio analyzer 140 receives biophysical sensor data 112A from a sensor 110A, biophysical sensor data 112B from a sensor 110B, biophysical sensor data 112C from a sensor 110C, one or more additional sets of sensor data from one or more sensors, or a combination thereof. The biophysical sensor data 112A is indicative of a biophysical rhythm 154A of a person 101A. The biophysical sensor data 112B is indicative of a biophysical rhythm 154B of a person 101B. The biophysical sensor data 112C is indicative of a biophysical rhythm 154C of a person 101C.

In a particular aspect, the audio analyzer 140 selects a subset of the received biophysical sensor data based on the sensor data selection criterion 862. For example, the audio analyzer 140 determines that the biophysical sensor data 112A satisfies the sensor data selection criterion 862 based on determining that the sensor 110A is within a threshold distance of the one or more speakers 104, that the biophysical sensor data 112A is indicative of the biophysical rhythm 154A of the person 101A who is detected within a threshold distance of the one or more speakers 104, or both. In some examples, any biophysical sensor data that is received by the audio analyzer 140 satisfies the sensor data selection criterion 862. In a particular aspect, the sensor data selection criterion 862 is based on default data, a configuration setting, a user input, or a

In an example, the audio analyzer 140 selects the biophysical sensor data 112A and the biophysical sensor data 112B in response to determining that each of the biophysical sensor data 112A and the biophysical sensor data 112B satisfy the sensor data selection criterion 862. In an example, the audio analyzer 140 determines that the biophysical sensor data 112C is not selected because the biophysical sensor data 112C fails to satisfy the sensor data selection criterion 862.

Sensor data (e.g., the biophysical sensor data 112A and the biophysical sensor data 112B) associated with two persons satisfying the sensor data selection criterion 862 is described as an illustrative example. In other examples, biophysical sensor data associated with fewer than two persons or more than two persons can satisfy the sensor data selection criterion 862. Although the sensor 110A, the sensor 110B, and the sensor 110C are illustrated as separate sensors, in some examples at least two of the sensor 110A, the sensor 110B, and the sensor 110C can be a single sensor (e.g., a camera).

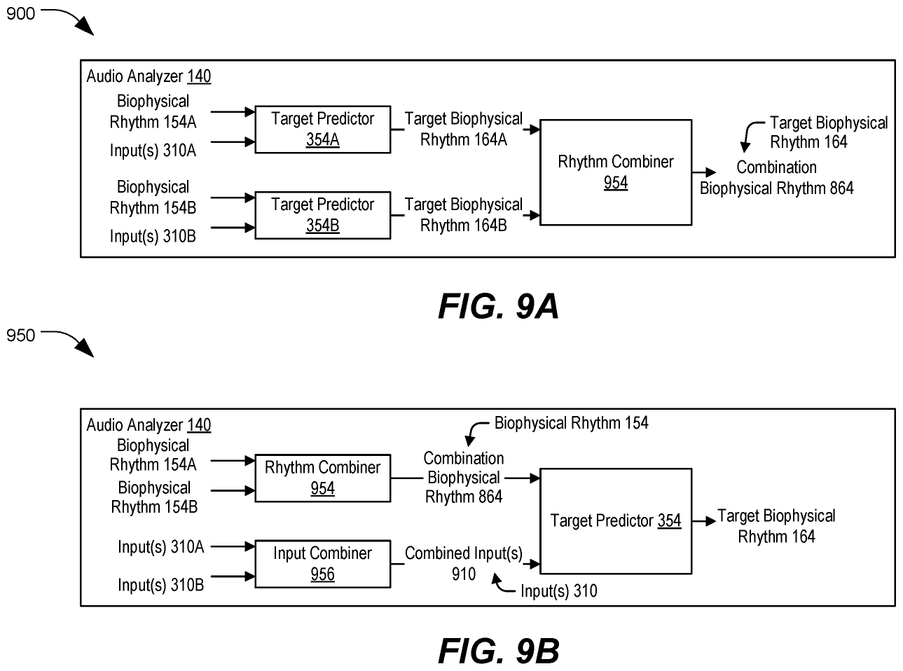

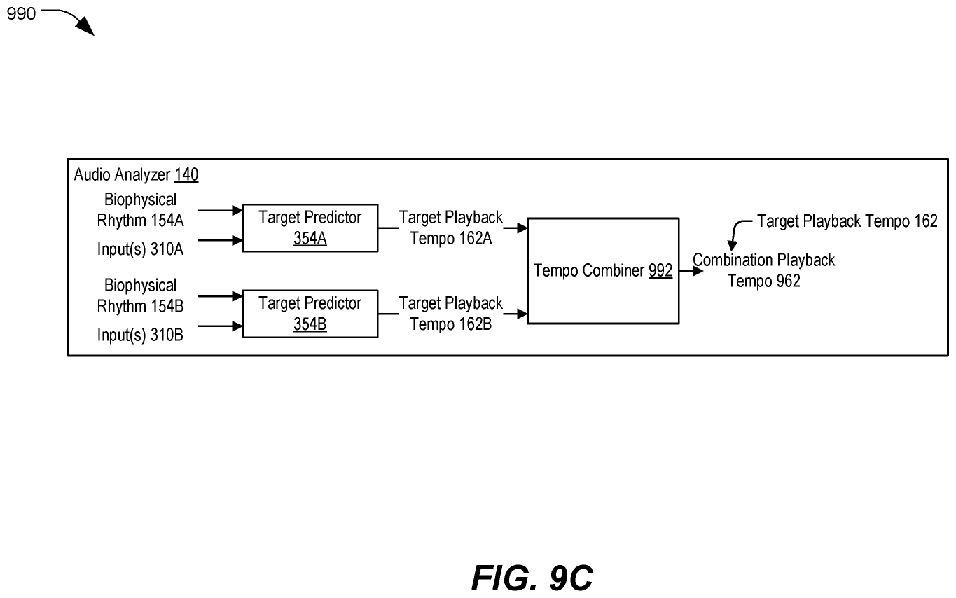

The audio analyzer 140, in response to determining that biophysical sensor data (e.g., the biophysical sensor data 112A) indicative of a biophysical rhythm of a single person satisfies the sensor data selection criterion 862, determines the target playback tempo 162 based on the corresponding biophysical rhythm (e.g., the biophysical rhythm 154A), as described with reference to FIGS. 1 and 3. Alternatively, the audio analyzer 140, in response to determining that biophysical sensor data (e.g., the biophysical sensor data 112A and the biophysical sensor data 112B) indicative of biophysical rhythms of multiple persons satisfies the sensor data selection criterion 862, determines a combination biophysical rhythm 864 based on corresponding biophysical rhythms (e.g., the biophysical rhythm 154A and the biophysical rhythm 154B), and determines the target playback tempo 162 based on the combination biophysical rhythm 864 as the target biophysical rhythm 164, as described with reference to FIGS. 9A-9B.

Optionally, in some implementations, the audio analyzer 140, in response to determining that biophysical sensor data (e.g., the biophysical sensor data 112A and the biophysical sensor data 112B) indicative of biophysical rhythms of multiple persons satisfies the sensor data selection criterion 862, determines the target playback tempo 162 (e.g., a combination playback tempo) based on corresponding biophysical rhythms (e.g., without an intermediate operation of determining the target biophysical rhythm 164), as described with reference to FIG. 9C.

In a particular aspect, the audio analyzer 140 updates the target playback tempo 162 based on a change in received biophysical sensor data 112 that satisfies the sensor data selection criterion 862. In an example, the change can correspond to biophysical sensor data (e.g., the biophysical sensor data 112B) no longer satisfying the sensor data selection criterion 862, additional biophysical sensor data (e.g., the biophysical sensor data 112C) satisfying the sensor data selection criterion 862, or both. The audio analyzer 140, in response to detecting the change, updates the target playback tempo 162 based on any biophysical sensor data (e.g., the biophysical sensor data 112A, the biophysical sensor data 112C, or both) that satisfies the sensor data selection criterion 862. If biophysical sensor data (e.g., the biophysical sensor data 112A) indicative of a biophysical rhythm of a single person satisfies the sensor data selection criterion 862, the audio analyzer 140 updates the target playback tempo 162 based on the corresponding biophysical rhythm (e.g., the biophysical rhythm 154A). If biophysical sensor data (e.g., the biophysical sensor data 112A and the biophysical sensor data 112C) indicative of biophysical rhythms of multiple persons satisfies the sensor data selection criterion 862, the audio analyzer 140 updates the combination biophysical rhythm 864 based on the corresponding biophysical rhythms and updates the target playback tempo 162 based on the updated version of the combination biophysical rhythm 864, as described with reference to FIGS. 9A-9B.

In an example, the change in received biophysical sensor data 112 can correspond to a greater than threshold change in the biophysical rhythm 154 indicated by the biophysical sensor data 112 that satisfies the sensor data selection criterion 862. For example, the audio analyzer 140 receives, at a first time, first biophysical sensor data 112A indicating a first biophysical rhythm 154A of the person 101A. The audio analyzer 140, in response to determining that the first biophysical sensor data 112A satisfies the sensor data selection criterion 862, determines the target playback tempo 162 based at least in part on the first biophysical rhythm 154A. The audio analyzer 140 receives, at a second time that is subsequent to the first time, second biophysical sensor data 112A indicative of a second biophysical rhythm 154A of the person 101A. The audio analyzer 140, in response to determining that the second biophysical sensor data 112A satisfies the sensor data selection criterion 862 and that a difference between the first biophysical rhythm 154A and the second biophysical rhythm 154A is greater than the threshold change, determines an updated target playback tempo 162 based at least in part on the second biophysical sensor data 112A. A technical advantage of determining the updated target playback tempo 162 based on a greater than threshold change in the biophysical rhythm 154 can include less frequent changes in the target playback tempo 162, thereby using fewer computing resources.

Referring to FIG. 9A, a diagram 900 of an illustrative aspect of operations associated with a rhythm combiner 954 is shown. The audio analyzer 140 includes a plurality of target predictors 354 coupled to the rhythm combiner 954.

The plurality of target predictors 354 include a target predictor 354A, a target predictor 354B, one or more additional target predictors, or a combination thereof. Each of the target predictors 354 generates a target biophysical rhythm 164, as described with reference to FIG. 3A. For example, the target predictor 354A processes the biophysical sensor data 112A, the biophysical rhythm 154A, the one or more inputs 310A, or a combination thereof, to generate a target biophysical rhythm 164A. As another example, the target predictor 354B processes the biophysical sensor data 112B, the biophysical rhythm 154B, the one or more inputs 310B, or a combination thereof to generate a target biophysical rhythm 164B.

The rhythm combiner 954 processes the target biophysical rhythms 164 from the target predictors 354 to generate the combination biophysical rhythm 864. For example, the combination biophysical rhythm 864 corresponds to an average (e.g., mean, median, or mode) of the target biophysical rhythm 164A and the target biophysical rhythm 164B. The combination biophysical rhythm 864 corresponds to the target biophysical rhythm 164. The audio analyzer 140 generates the target playback tempo 162 based on the target biophysical rhythm 164 (e.g., the combination biophysical rhythm 864), as described with reference to FIG. 1.

Referring to FIG. 9B, a diagram 950 of an illustrative aspect of operations associated with a rhythm combiner 954 is shown. The audio analyzer 140 includes the rhythm combiner 954 coupled to the target predictor 354. Optionally, in some implementations, the audio analyzer 140 also includes an input combiner 956 coupled to the target predictor 354.

The rhythm combiner 954 processes the biophysical rhythms 154 to generate the combination biophysical rhythm 864. For example, the combination biophysical rhythm 864 corresponds to an average (e.g., mean, median, or mode) of the biophysical rhythm 154A and the biophysical rhythm 154B. The combination biophysical rhythm 864 corresponds to the biophysical rhythm 154 that is processed by the target predictor 354 to generate the target biophysical rhythm 164, as described with reference to FIG. 3A.

Optionally, in some implementations, the input combiner 956 generates one or more combined inputs 910 based on one or more inputs 310 associated with the biophysical rhythms 154. For example, one or more inputs 310A are associated with the biophysical rhythm 154A. As another example, one or more inputs 310B are associated with the biophysical rhythm 154B. The input combiner 956 generates the one or more combined inputs 910 based on the one or more inputs 310A and the one or more inputs 310B.

In a particular aspect, a particular combined input included in the one or more combined inputs 910 corresponds to an average (e.g., mean, median, or mode) based on a corresponding input of the one or more inputs 310A and a corresponding input of the one or more inputs 310B. For example, the one or more inputs 310A include a first calorie target 314, and the one or more inputs 310B include a second calorie target 314. In this example, the one or more combined inputs 910 can include a combined calorie target 314 corresponding to an average calorie target (e.g., mean, median, or mode) based on the first calorie target 314 and the second calorie target 314. The target predictor 354 processes the combination biophysical rhythm 864 (as the biophysical rhythm 154), the one or more combined inputs 910 (as the one or more inputs 310), or a combination thereof, to generate the target biophysical rhythm 164, as described with reference to FIG. 3A. The audio analyzer 140 generates the target playback tempo 162 based on the target biophysical rhythm 164, as described with reference to FIG. 1.

Referring to FIG. 9C, a diagram 990 of an illustrative aspect of operations associated with a tempo combiner 992 is shown. The audio analyzer 140 includes a plurality of target predictors 354 coupled to the tempo combiner 992.

Each of the target predictors 354 generates a target playback tempo 162, as described with reference to FIG. 3B. For example, the target predictor 354A processes the biophysical sensor data 112A, the biophysical rhythm 154A, the one or more inputs 310A, or a combination thereof, to generate a target playback tempo 162A. As another example, the target predictor 354B processes the biophysical sensor data 112B, the biophysical rhythm 154B, the one or more inputs 310B, or a combination thereof, to generate a target playback tempo 162B.

The tempo combiner 992 processes the target playback tempos 162 from the target predictors 354 to generate a combination playback tempo 962 as the target playback tempo 162. For example, the combination playback tempo 962 corresponds to an average (e.g., mean, median, or mode) of the target playback tempo 162A and the target playback tempo 162B.

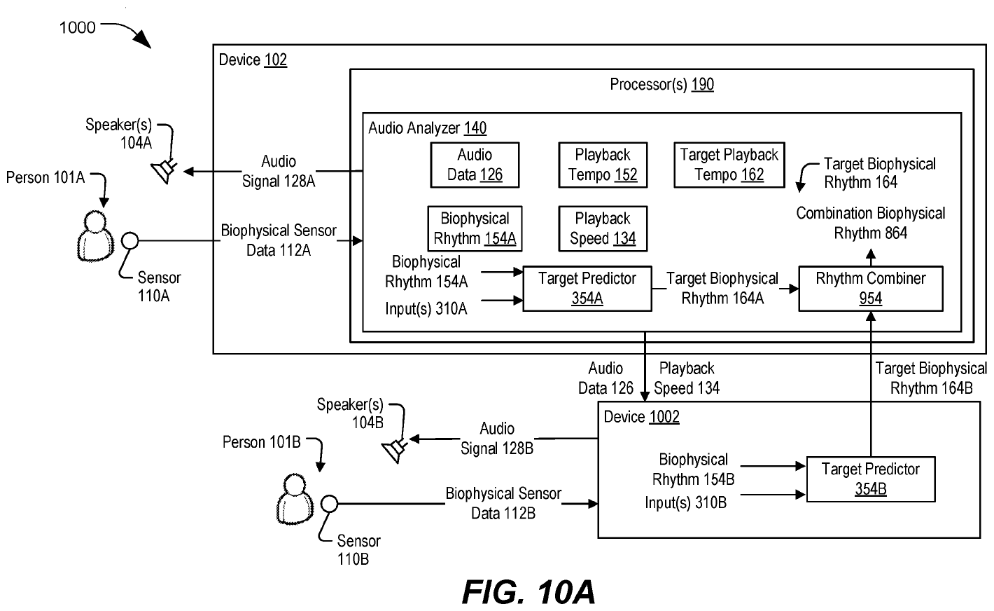

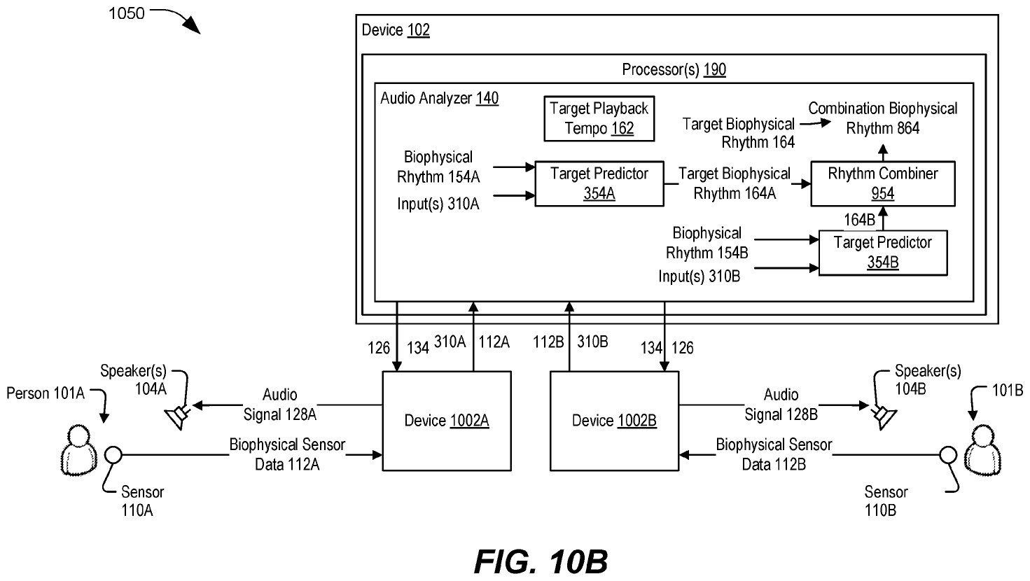

Referring to FIG. 10A, a diagram is shown of an illustrative implementation of a system 1000 operable to adjust audio playback speed based at least in part on a target biophysical rhythm determined at another device. In a particular aspect, the system 100 of FIG. 1 includes one or more components of the system 1000.

The device 102 is communicatively coupled to one or more devices, such as a device 1002. The device 1002 includes the target predictor 354B that generates the target biophysical rhythm 164B based on the biophysical sensor data 112B, the biophysical rhythm 154B, the one or more inputs 310B, or a combination thereof, as described with reference to FIG. 3A.

The audio analyzer 140 determines the target playback tempo 162 based on target biophysical rhythms received from one or more other devices. For example, the audio analyzer 140 determines the target playback tempo 162 based at least in part on the target biophysical rhythm 164B received from the device 1002, one or more additional target biophysical rhythms received from one or more devices, or a combination thereof, as described with reference to FIGS. 9A-9B.

In some implementations, the device 102 includes the target predictor 354A that generates the target biophysical rhythm 164A based on the biophysical sensor data 112A, the biophysical rhythm 154A, the one or more inputs 310A, or a combination thereof, as described with reference to FIG. 3A. The audio analyzer 140 determines the target playback tempo 162 also based on the target biophysical rhythm 164A.

Optionally, in some implementations, the audio analyzer 140 determines the target playback tempo 162 based on target playback tempos received from one or more devices. For example, the target predictor 354B generates the target playback tempo 162B based on the biophysical sensor data 112B, the biophysical rhythm 154B, the one or more inputs 310B, or a combination thereof, as described with reference to FIG. 3B. The target predictor 354B of the device 1002 provides the target playback tempo 162A to the audio analyzer 140 of the device 102. The audio analyzer 140 generates the target playback tempo 162 based on the target playback tempo 162B, one or more additional target playback tempos received from one or more devices, or a combination thereof, as described with reference to FIG. 9C. In some implementations, the target predictor 354A generates the target playback tempo 162A based on the biophysical sensor data 112A, the biophysical rhythm 154A, the one or more inputs 310A, or a combination thereof, as described with reference to FIG. 3B. The audio analyzer 140 determines the target playback tempo 162 also based on the target playback tempo 162A.

The audio analyzer 140 determines the playback speed 134 based on the playback tempo 152 and the target playback tempo 162, as described with reference to FIG. 1. The audio analyzer 140 provides the audio data 126, the playback speed 134, or both, to the device 1002. The device 1002 outputs, via one or more speakers 104B, an audio signal 128B corresponding to the audio data 126 having the playback speed 134. In some implementations, the audio analyzer 140 also outputs, via one or more speakers 104A, an audio signal 128A corresponding to the audio data 126 having the playback speed 134.

A technical advantage of off-loading operations to determine the target biophysical rhythm 164B (or the target playback tempo 162B) from the device 102 to the device 1002 includes improved efficiency (e.g., faster, fewer computing cycles, or both) at the device 102. Additionally, the person 101A and the person 101B can have a shared experience of the audio data 126 having the same playback speed while using different playback devices (e.g., the device 102 and the device 1002).

In some implementations, the audio analyzer 140 can select distinct audio data for playback by each of the device 102 and the device 1002. For example, the audio analyzer 140 can select the audio data 126A, for playback by the device 102, based on the playback tempo 152A, the target playback tempo 162, a detected mood, a target mood, a user preference, a user playlist, an age restriction, an audio service membership, a cost associated with providing the audio data 126A to the person 101A, or a combination thereof, as described with reference to FIG. 5C. Similarly, the audio analyzer 140 can select the audio data 126B, for playback by the device 1002, based on the playback tempo 152B, the target playback tempo 162, a detected mood, a target mood, a user preference, a user playlist, an age restriction, an audio service membership, a cost associated with providing the audio data 126B to the person 101B, or a combination thereof.

The audio analyzer 140 determines a first playback speed 134 based on the playback tempo 152A and the target playback tempo 162 (e.g., the first playback speed 134=the target playback tempo 162/the playback tempo 152A), and a second playback speed 134 based on the playback tempo 152B and the target playback tempo 162 (e.g., the second playback speed 134−the target playback tempo 162/the playback tempo 152B). The audio analyzer 140 outputs the audio signal 128A via the one or more speakers 104A. The audio signal 128A corresponds to the audio data 126A at the first playback speed 134 having the target playback tempo 162. The audio analyzer 140 provides the audio data 126B, the second playback speed 134, or both, to the device 1002. The device 1002 outputs the audio signal 128B via the one or more speakers 104B. The audio signal 128B corresponds to the audio data 126B at the second playback speed 134 having the target playback tempo 162.

In these implementations, a technical advantage can include the person 101A and the person 101B having a shared experience of audio having the same playback tempo while using different playback devices (e.g., the device 102 and the device 1002) to listen to different audio data (e.g., the audio data 126A and the audio data 126B).

Referring to FIG. 10B, a diagram is shown of an illustrative implementation of a system 1050 operable to adjust audio playback speed based on biophysical sensor data received from another device. In a particular aspect, the system 100 of FIG. 1 includes one or more components of the system 1050.

The device 102 is communicatively coupled to one or more devices, such as a device 1002A, a device 1002B, one or more additional devices, or a combination thereof. The device 1002A provides the biophysical sensor data 112A, the one or more inputs 310A, or a combination thereof, to the audio analyzer 140 of the device 102. The device 1002B provides the biophysical sensor data 112B, the one or more inputs 310B, or a combination thereof, to the audio analyzer 140 of the device 102.

The audio analyzer 140 determines the target playback tempo 162 based on biophysical sensor data received from one or more other devices. For example, the audio analyzer 140 determines the target playback tempo 162 based at least in part on the biophysical sensor data 112A received from the device 1002A, the biophysical sensor data 112B received from the device 1002B, additional biophysical sensor data received from one or more other devices, or a combination thereof, as described with reference to FIGS. 9A-9C.

In a particular implementation, the audio analyzer 140 determines target biophysical rhythms based on biophysical sensor data received from one or more other devices. For example, the device 102 includes the target predictor 354A that generates the target biophysical rhythm 164A based on the biophysical sensor data 112A, the biophysical rhythm 154A, the one or more inputs 310A, or a combination thereof, as described with reference to FIG. 3A. As another example, the device 102 includes the target predictor 354B that generates the target biophysical rhythm 164B based on the biophysical sensor data 112B, the biophysical rhythm 154B, the one or more inputs 310B, or a combination thereof, as described with reference to FIG. 3A. In this implementation, the rhythm combiner 954 generates the combination biophysical rhythm 864 based on the target biophysical rhythm 164A, the target biophysical rhythm 164B, one or more additional target biophysical rhythms, or a combination thereof, as described with reference to FIG. 9A. The combination biophysical rhythm 864 corresponds to the target biophysical rhythm 164. The audio analyzer 140 determines the target playback tempo 162 based on the target biophysical rhythm 164, as described with reference to FIG. 1.

The audio analyzer 140 including the target predictors 354 configured to generate target biophysical rhythms 164 and including the rhythm combiner 954 configured to generate the combination biophysical rhythm 864 as the target biophysical rhythm 164 is provided as an illustrative implementation. Optionally, in some implementations, the audio analyzer 140 determines a combination biophysical rhythm based on biophysical sensor data received from one or more other devices. For example, the device 102 includes the rhythm combiner 954 that generates the combination biophysical rhythm 864 based on the biophysical sensor data 112A, the biophysical rhythm 154A, the biophysical sensor data 112B, the biophysical rhythm 154B, or a combination thereof, as described with reference to FIG. 9B. The device 102 can also include the input combiner 956 that generates the one or more combined inputs 910 based on the one or more inputs 310A, the one or more inputs 310B, or a combination thereof, as described with reference to FIG. 9B. In this implementation, the audio analyzer 140 includes the target predictor 354 that determines the target biophysical rhythm 164 based on the combination biophysical rhythm 864 (as the biophysical rhythm 154), the one or more combined inputs 910 (e.g., as the one or more inputs 310), or a combination thereof, as described with reference to FIG. 9B. The audio analyzer 140 determines the target playback tempo 162 based on the target biophysical rhythm 164, as described with reference to FIG. 1.

Optionally, in some implementations, the audio analyzer 140 determines a combination playback tempo based on biophysical sensor data received from one or more other devices. For example, the device 102 includes the target predictor 354A that generates the target playback tempo 162A based on the biophysical sensor data 112A, the biophysical rhythm 154A, the one or more inputs 310A, or a combination thereof, as described with reference to FIG. 3B. As another example, the device 102 includes the target predictor 354B that generates the target playback tempo 162B based on the biophysical sensor data 112B, the biophysical rhythm 154B, the one or more inputs 310B, or a combination thereof, as described with reference to FIG. 3B. In this implementation, the audio analyzer 140 includes the tempo combiner 992 that generates the combination playback tempo 962 as the target playback tempo 162, as described with reference to FIG. 9C.

The audio analyzer 140 determines the playback speed 134 based on the playback tempo 152 and the target playback tempo 162, as described with reference to FIG. 1. The audio analyzer 140 provides the audio data 126, the playback speed 134, or both, to the device 1002A, the device 1002B, or both. The device 1002A outputs, via one or more speakers 104A, an audio signal 128A corresponding to the audio data 126 having the playback speed 134. Similarly, the device 1002B outputs, via one or more speakers 104B, an audio signal 128B corresponding to the audio data 126 having the playback speed 134.

A technical advantage can include the operations to determine the target playback tempo 162 being performed at the device 102 (e.g., a server) instead of being duplicated at each of the one or more devices 1002. Additionally, the person 101A and the person 101B can have a shared experience of the audio data 126 having the same playback speed while using different playback devices (e.g., the device 1002A and the device 1002B).

In some implementations, the audio analyzer 140 can select distinct audio data for playback by each of the device 1002A and the device 1002B. For example, the audio analyzer 140 can select the audio data 126A for playback by the device 1002A, as described with reference to FIGS. 5C and 10A. Similarly, the audio analyzer 140 can select the audio data 126B for playback by the device 1002B.

The audio analyzer 140 determines a first playback speed 134 based on the playback tempo 152A and the target playback tempo 162, and a second playback speed 134 based on the playback tempo 152B and the target playback tempo 162. The audio analyzer 140 provides the audio data 126A, the first playback speed 134, or both, to the device 1002A, and provides the audio data 126B, the second playback speed 134, or both, to the device 1002B. The device 1002A outputs the audio signal 128A via the one or more speakers 104A, and the device 1002B outputs the audio signal 128B via the one or more speakers 104B. The audio signal 128A corresponds to the audio data 126A at the first playback speed 134 having the target playback tempo 162. The audio signal 128B corresponds to the audio data 126B at the second playback speed 134 having the target playback tempo 162.

In these implementations, a technical advantage can include the person 101A and the person 101B having a shared experience of audio having the same playback tempo while using different playback devices (e.g., the device 1002A and the device 1002B) to listen to different audio data (e.g., the audio data 126A and the audio data 126B).

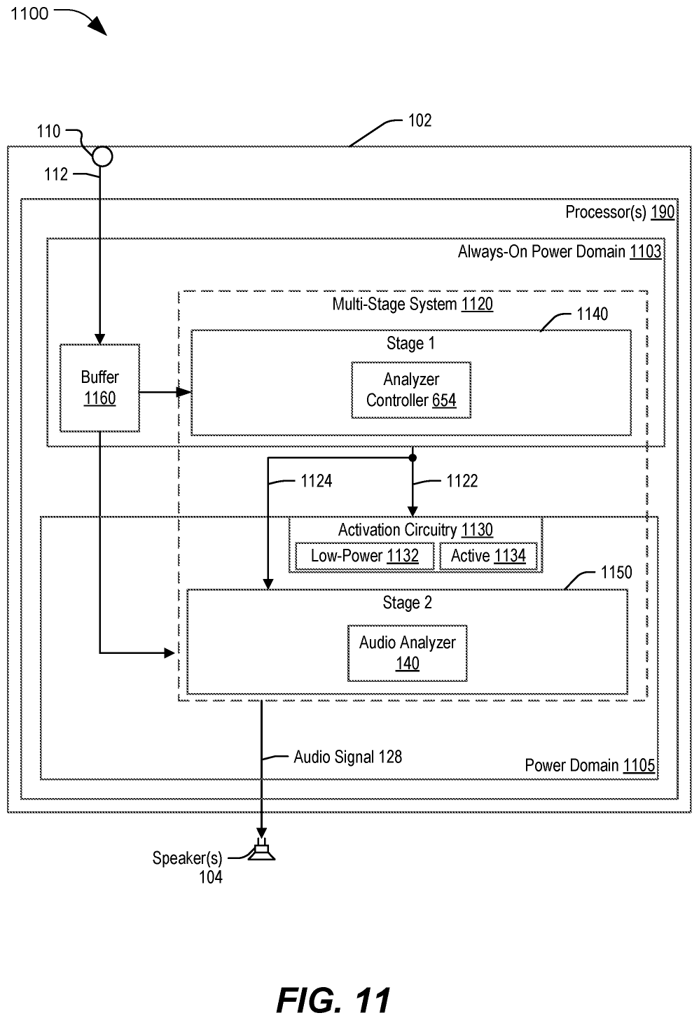

FIG. 11 is a block diagram of an illustrative aspect of a system operable to perform audio playback speed adjustment, in accordance with some examples of the present disclosure, in which the one or more processors 190 include an always-on power domain 1103 and a second power domain 1105, such as an on-demand power domain.

In some implementations, a first stage 1140 of a multi-stage system 1120 and a buffer 1160 are configured to operate in an always-on mode, and a second stage 1150 of the multi-stage system 1120 is configured to operate in an on-demand mode.

The always-on power domain 1103 includes the buffer 1160 and the first stage 1140 including the analyzer controller 654. In a particular aspect, the buffer 1160 includes the input audio buffer 564 of FIG. 5A. The buffer 1160 is configured to store the audio data 126 of FIG. 1, the images 220 of FIG. 2, the input audio signal 503 of FIG. 5, the user input 626 of FIG. 6, or a combination thereof, to be accessible for processing by components of the multi-stage system 1120.

The second power domain 1105 includes the second stage 1150 of the multi-stage system 1120 and also includes activation circuitry 1130.

The first stage 1140 of the multi-stage system 1120 is configured to generate at least one of a wakeup signal 1122 or an interrupt 1124 to initiate one or more operations at the second stage 1150. In an example, the wakeup signal 1122 is configured to transition the second power domain 1105 from a low-power mode 1132 to an active mode 1134 to activate one or more components of the second stage 1150.

For example, the activation circuitry 1130 may include or be coupled to power management circuitry, clock circuitry, head switch or foot switch circuitry, buffer control circuitry, or any combination thereof. The activation circuitry 1130 may be configured to initiate powering-on of the second stage 1150, such as by selectively applying or raising a voltage of a power supply of the second stage 1150, of the second power domain 1105, or both. As another example, the activation circuitry 1130 may be configured to selectively gate or un-gate a clock signal to the second stage 1150, such as to prevent or enable circuit operation without removing a power supply.

In a particular aspect, the first stage 1140 includes the analyzer controller 654 that is configured to generate the start command 638, as described with reference to FIG. 6. In a particular aspect, the start command 638 corresponds to at least one of the wakeup signal 1122 or the interrupt 1124.

The audio signal 128 generated by the second stage 1150 of the multi-stage system 1120 is provided to the one or more speakers 104. In a particular aspect, the audio signal 128 is provided to an application. For example, the application may correspond to an exercise application, a playback application, a voice interface application, an integrated assistant application, a vehicle navigation and entertainment application, or a home automation system, as illustrative, non-limiting examples.

A technical advantage of selectively activating the second stage 1150 based on detecting the playback condition 628 of FIG. 6 at the first stage 1140 of the multi-stage system 1120 can include a reduction in overall power consumption associated with audio playback speed adjustment.

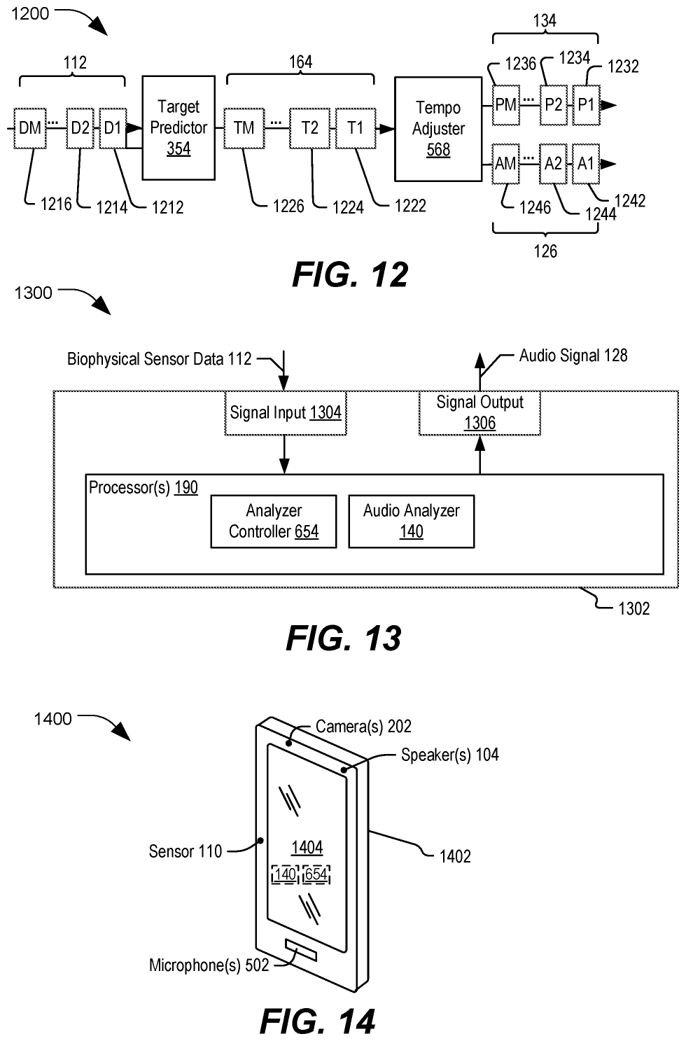

FIG. 12 is a diagram of an illustrative aspect of operation of components of the system of FIG. 1, in accordance with some examples of the present disclosure. The target predictor 354 is configured to receive the biophysical sensor data 112, such as a sequence of successively captured values of the biophysical sensor data 112, illustrated as first sensor data (D1) 1212, second sensor data (D2) 1214, and one or more additional values of sensor data including Mth sensor data (DM) 1216 (where M is an integer greater than two). The target predictor 354 is configured to output values of the target biophysical rhythm 164, such as a sequence of values of the target biophysical rhythm 164 including a first target biophysical rhythm (T1) 1222, a second target biophysical rhythm (T2) 1224, and one or more additional values including an Mth target biophysical rhythm (TM) 1226.

The tempo adjuster 568 is configured to receive the target biophysical rhythm 164, such as a sequence of values of the target biophysical rhythm 164, and to adaptively adjust playback speed of audio data.

During operation, the target predictor 354 processes the first sensor data (D1) 1212 to determine the first target biophysical rhythm (T1) 1222. The tempo adjuster 568 determines a playback speed (P1) 1232 based at least in part on the first target biophysical rhythm (T1) 1222 and adjusts the audio data 126 to generate a first set of audio frames (A1) 1242 corresponding to the playback speed (P1) 1232. The target predictor 354 processes the second sensor data (D2) 1214 to determine the second target biophysical rhythm (T2) 1224. The tempo adjuster 568 determines a playback speed (P2) 1234 based at least in part on the second target biophysical rhythm (T2) 1224 and adjusts the audio data 126 to generate a second set of audio frames (A2) 1244 corresponding to the playback speed (P2) 1234. Such processing continues, including the target predictor 354 processing the Mth sensor data (DM) 1216 to determine the Mth target biophysical rhythm (TM) 1226. The tempo adjuster 568 determines a playback speed (PM) 1236 based at least in part on the Mth target biophysical rhythm (TM) 1226 and adjusts the audio data 126 to generate an Mth set of audio frames (AM) 1246 corresponding to the playback speed (PM) 1236.

The target biophysical rhythm can thus be dynamically adjusted based at least in part on changes in the biophysical sensor data. The playback speed of the audio data can be automatically adjusted to correspond to changes in the target biophysical rhythm.

FIG. 13 depicts an implementation 1300 of the device 102 as an integrated circuit 1302 that includes the one or more processors 190. The one or more processors 190 include the audio analyzer 140, the analyzer controller 654, or both. The integrated circuit 1302 also includes a signal input 1304, such as one or more bus interfaces, to enable the biophysical sensor data 112 to be received for processing. The integrated circuit 1302 also includes a signal output 1306, such as a bus interface, to enable sending of the audio signal 128, the audio data 126, the playback speed 134, or a combination thereof. The integrated circuit 1302 enables implementation of audio playback speed adjustment as a component in a system, such as a mobile phone or tablet as depicted in FIG. 14, a headset device as depicted in FIG. 15, earbuds as depicted in FIG. 16, a wearable electronic device as depicted in FIG. 17, extended reality glasses as depicted in FIG. 18, a voice-controlled speaker system as depicted in FIG. 19, a virtual reality, mixed reality, or augmented reality headset, as depicted in FIG. 20, or a vehicle as depicted in FIG. 21 or FIG. 22.

FIG. 14 depicts an implementation 1400 in which the device 102 includes a mobile device 1402, such as a phone or tablet, as illustrative, non-limiting examples. The mobile device 1402 includes the one or more speakers 104, the one or more microphones 502, one or more cameras 202, and a display screen 1404. In a particular aspect, the mobile device 1402 includes the sensor 110. In some implementations, the sensor 110 includes the one or more cameras 202.

Components of the one or more processors 190, including the audio analyzer 140, the analyzer controller 654, or both, are integrated in the mobile device 1402 and are illustrated using dashed lines to indicate internal components that are not generally visible to a user of the mobile device 1402. In a particular example, the analyzer controller 654 operates to detect user voice activity as the playback condition 628, which is then processed to perform one or more operations at the mobile device 1402, such as to launch a graphical user interface or otherwise display other information associated with the user's speech at the display screen 1404 (e.g., via an integrated “smart assistant” application). For example, the display screen 1404 can indicate when the audio analyzer 140 is activated. In a particular implementation, the audio analyzer 140 displays the GUI 501 of FIG. 5A at the display screen 1404. In some examples, the audio analyzer 140 outputs, via the one or more speakers 104, the audio signal 128 (e.g., music) based on the biophysical sensor data 112 from the sensor 110.

FIG. 15 depicts an implementation 1500 in which the device 102 includes a headset device 1502. The headset device 1502 includes the one or more speakers 104, the one or more microphones 502, the one or more cameras 202, the sensor 110, or a combination thereof. In a particular aspect, the sensor 110 includes the one or more cameras 202. In a particular aspect, the sensor 110 includes a heartrate monitor. Components of the one or more processors 190, including the audio analyzer 140, the analyzer controller 654, or both, are integrated in the headset device 1502. In a particular example, the analyzer controller 654 operates to detect user voice activity as the playback condition 628, which may cause the headset device 1502 to perform one or more operations at the headset device 1502, such as to activate the audio analyzer 140 and to provide the audio signal 128 to the one or more speakers 104, a second device (not shown), or both, for playback.

FIG. 16 depicts an implementation 1600 in which the device 102 includes a portable electronic device that corresponds to a pair of earbuds 1606 that includes a first earbud 1602 and a second earbud 1604. Although earbuds are described, it should be understood that the present technology can be applied to other in-ear or over-ear playback devices.

The first earbud 1602 includes a first microphone 1620, such as a high signal-to-noise microphone positioned to capture the voice of a wearer of the first earbud 1602, an array of one or more other microphones configured to detect ambient sounds and spatially distributed to support beamforming, illustrated as microphones 1622A, 1622B, and 1622C, an “inner” microphone 1624 proximate to the wearer's ear canal (e.g., to assist with active noise cancelling), and a self-speech microphone 1626, such as a bone conduction microphone configured to convert sound vibrations of the wearer's ear bone or skull into an audio signal. In some aspects, the first earbud 1602 includes a sensor 110 configured to generate the biophysical sensor data 112.

In a particular implementation, the one or more microphones 502 of FIG. 5C correspond to the microphones 1620, 1622A, 1622B, and 1622C, and audio signals generated by the microphones 1620 and 1622A, 1622B, and 1622C are provided to the audio analyzer 140, the analyzer controller 654, or both. The analyzer controller 654 may function to generate the start command 638 or the stop command 640 based on the audio signals. In some examples, the audio analyzer 140 may function to determine the detected mood 532 based on the audio signals. In some implementations, the audio analyzer 140, the analyzer controller 654, or both, may further be configured to process audio signals from one or more other microphones of the first earbud 1602, such as the inner microphone 1624, the self-speech microphone 1626, or both.

The first earbud 1602 includes a speaker 1630. In some aspects, the speaker 1630 corresponds to the one or more speakers 104 of FIG. 1. For example, the audio analyzer 140 outputs the audio signal 128 via the speaker 1630.

The second earbud 1604 can be configured in a substantially similar manner as the first earbud 1602. In some implementations, the audio analyzer 140, the analyzer controller 654, or both, of the first earbud 1602 are also configured to receive one or more audio signals generated by one or more microphones of the second earbud 1604, such as via wireless transmission between the earbuds 1602, 1604, or via wired transmission in implementations in which the earbuds 1602, 1604 are coupled via a transmission line. In other implementations, the second earbud 1604 also includes an audio analyzer 140, an analyzer controller 654, or both, enabling techniques described herein to be performed by a user wearing a single one of either of the earbuds 1602, 1604.

In some implementations, the earbuds 1602, 1604 are configured to automatically switch between various operating modes, such as a passthrough mode in which ambient sound is played via the speaker 1630, a playback mode in which non-ambient sound (e.g., streaming audio corresponding to a phone conversation, media playback, video game, etc.) is played back through the speaker 1630, and an audio zoom mode or beamforming mode in which one or more ambient sounds are emphasized and/or other ambient sounds are suppressed for playback at the speaker 1630. In other implementations, the earbuds 1602, 1604 may support fewer modes or may support one or more other modes in place of, or in addition to, the described modes.

In an illustrative example, the earbuds 1602, 1604 can automatically transition from the playback mode to the passthrough mode in response to detecting the wearer's voice, and may automatically transition back to the playback mode after the wearer has ceased speaking. In some examples, the earbuds 1602, 1604 can operate in two or more of the modes concurrently, such as by performing audio zoom on a particular ambient sound (e.g., a dog barking) and playing out the audio zoomed sound superimposed on the sound being played out while the wearer is listening to music (which can be reduced in volume while the audio zoomed sound is being played). In this example, the wearer can be alerted to the ambient sound associated with the audio event without halting playback of the music.

FIG. 17 depicts an implementation 1700 in which the device 102 includes a wearable electronic device 1702, illustrated as a “smart watch.” The audio analyzer 140, the analyzer controller 654, the one or more speakers 104, the one or more cameras 202, the one or more microphones 502, the sensor 110, or a combination thereof, are integrated into the wearable electronic device 1702.

In a particular example, the analyzer controller 654 operates to detect user voice activity, which is then processed to perform one or more operations at the wearable electronic device 1702, such as to launch a graphical user interface or otherwise display other information associated with the user's speech at a display screen 1704 of the wearable electronic device 1702. To illustrate, the wearable electronic device 1702 may include a display screen that is configured to display a notification based on user speech detected by the wearable electronic device 1702. For example, the notification indicates that the audio analyzer 140 is activated.

In a particular example, the wearable electronic device 1702 includes a haptic device that provides a haptic notification (e.g., vibrates) in response to detection of user voice activity. For example, the haptic notification can cause a user to look at the wearable electronic device 1702 to see a displayed notification indicating detection of a keyword spoken by the user. The wearable electronic device 1702 can thus alert a user with a hearing impairment or a user wearing a headset that the user's voice activity is detected. In some examples, the displayed notification can include the GUI 501 of FIG. 5A.

FIG. 18 depicts an implementation 1800 in which the device 102 includes a portable electronic device that corresponds to extended reality (XR) glasses 1802. The glasses 1802 include a holographic projection unit 1804 configured to project visual data onto a surface of a lens 1806 or to reflect the visual data off of a surface of the lens 1806 and onto the wearer's retina. The audio analyzer 140, the analyzer controller 654, the one or more speakers 104, the one or more cameras 202, the one or more microphones 502, the sensor 110, or a combination thereof, are integrated into the glasses 1802. The analyzer controller 654 may function to generate the start command 638 or the stop command 640 based on audio signals received from the one or more microphones 502. In a particular example, the holographic projection unit 1804 is configured to display a notification indicating user speech detected in the audio signal. In a particular example, the holographic projection unit 1804 is configured to display a notification indicating a detected audio event. For example, the notification can be superimposed on the user's field of view at a particular position that coincides with the location of the source of the sound associated with the audio event. To illustrate, the sound may be perceived by the user as emanating from the direction of the notification. In an illustrative implementation, the holographic projection unit 1804 is configured to display a notification of activation of the audio analyzer 140. In some examples, the holographic projection unit 1804 is configured to display the GUI 501 of FIG. 5A.

FIG. 19 is an implementation 1900 in which the device 102 includes a wireless speaker and voice activated device 1902. The wireless speaker and voice activated device 1902 can have wireless network connectivity and is configured to execute an assistant operation. The one or more processors 190 including the audio analyzer 140, the analyzer controller 654, or both, are included in the wireless speaker and voice activated device 1902. In a particular aspect, the one or more cameras 202, the one or more microphones 502, the sensor 110, or a combination thereof, are included in the wireless speaker and voice activated device 1902. The wireless speaker and voice activated device 1902 also includes the one or more speakers 104.

During operation, in response to receiving a verbal command identified as user speech via operation of the analyzer controller 654, the wireless speaker and voice activated device 1902 can execute playback operations, such as via execution of the audio analyzer 140. For example, the audio playback speed adjustment is performed responsive to receiving a command after a keyword or key phrase (e.g., “hello assistant”).

FIG. 20 depicts an implementation 2000 in which the device 102 includes a portable electronic device that corresponds to a virtual reality, mixed reality, or augmented reality headset 2002. The audio analyzer 140, the analyzer controller 654, the one or more speakers 104, the sensor 110, the one or more cameras 202, the one or more microphones 502, or a combination thereof, are integrated into the headset 2002. User voice activity detection can be performed based on audio signals received from the one or more microphones 502 of the headset 2002. A visual interface device is positioned in front of the user's eyes to enable display of augmented reality, mixed reality, or virtual reality images or scenes to the user while the headset 2002 is worn. In a particular example, the visual interface device is configured to display a notification indicating user speech detected in the audio signal. In some examples, the visual interface device is configured to display a notification indicating that the audio analyzer 140 is activated. In some examples, the visual interface device is configured to display the GUI 501 of FIG. 5A.

FIG. 21 depicts an implementation 2100 in which the device 102 corresponds to, or is integrated within, a vehicle 2102, illustrated as a manned or unmanned aerial device (e.g., a package delivery drone). The audio analyzer 140, the analyzer controller 654, the one or more speakers 104, the sensor 110, the one or more cameras 202, the one or more microphones 502, or a combination thereof, are integrated into the vehicle 2102. User voice activity detection can be performed based on audio signals received from the one or more microphones 502 of the vehicle 2102, such as for playback instructions from an authorized user of the vehicle 2102.

FIG. 22 depicts another implementation 2200 in which the device 102 corresponds to, or is integrated within, a vehicle 2202, illustrated as a car. The vehicle 2202 includes the one or more processors 190 including the audio analyzer 140, the analyzer controller 654, or both. The vehicle 2202 also includes the one or more speakers 104, the sensor 110, the one or more cameras 202, the one or more microphones 502, or a combination thereof. User voice activity detection can be performed based on audio signals received from the one or more microphones 502 of the vehicle 2202. In some implementations, user voice activity detection can be performed based on an audio signal received from interior microphones (e.g., at least one of the one or more microphones 502), such as for a voice command from an authorized passenger. For example, the user voice activity detection can be used to detect a voice command from an operator of the vehicle 2202 (e.g., a voice command from a parent to automatically adjust playback audio speed) and to disregard the voice of another passenger (e.g., a voice command from a child to deactivate the playback audio speed adjustment). In some implementations, user voice activity detection can be performed based on an audio signal received from external microphones (e.g., at least one of the one or more microphones 502), such as an authorized user of the vehicle. In a particular implementation, in response to receiving a verbal command identified as user speech via operation of the analyzer controller 654, a voice activation system activates the audio analyzer 140 of the vehicle 2202 based on one or more detected keywords (e.g., “auto adjust playback speed” or another voice command), such as by providing feedback or information (e.g., the GUI 501 of FIG. 5A) via a display 2220 or providing the audio signal 128 via the one or more speakers 104.

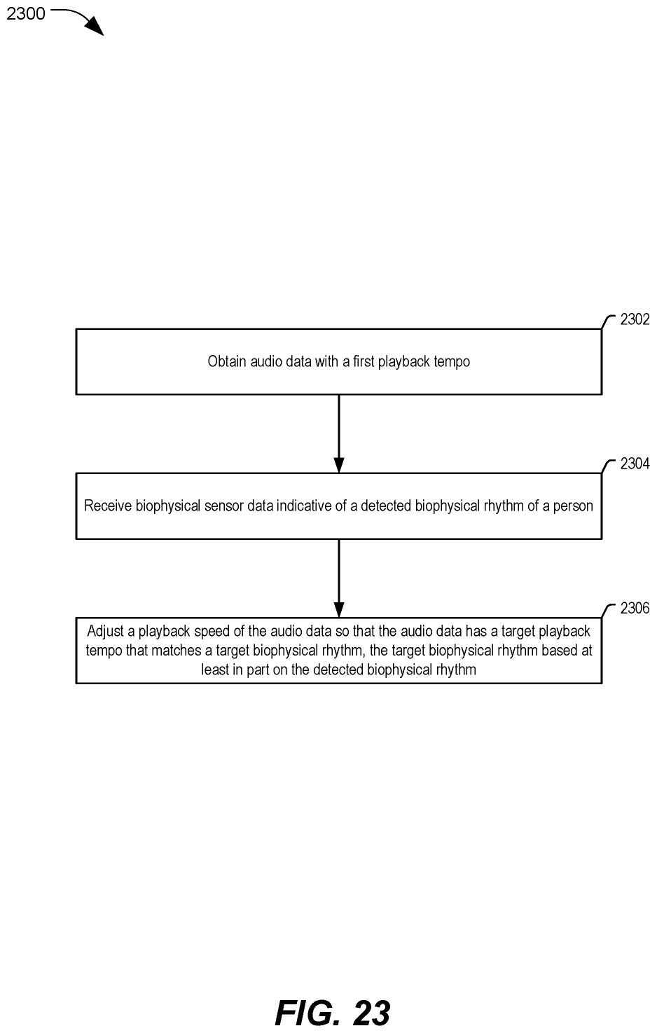

Referring to FIG. 23, a particular implementation of a method 2300 of audio playback speed adjustment is shown. In a particular aspect, one or more operations of the method 2300 are performed by at least one of the audio analyzer 140, the one or more processors 190, the device 102, the system 100 of FIG. 1, the input audio buffer 564, the tempo estimator 566, the tempo adjuster 568, the system 500 of FIG. 5A, the analyzer controller 654, the system 600 of FIG. 6, the system 700 of FIG. 7, the system 800 of FIG. 8, the system 1000 of FIG. 10A, the system 1050 of FIG. 10B, or a combination thereof.

The method 2300 includes, at 2302, obtaining audio data with a first playback tempo. For example, the audio analyzer 140 of FIG. 1 obtains the audio data 126 having the playback tempo 152, as described with reference to FIGS. 1 and 5A-5C.

The method 2300 also includes, at 2304, receiving biophysical sensor data indicative of a detected biophysical rhythm of a person. For example, the audio analyzer 140 of FIG. 1 receives the biophysical sensor data 112 indicative of the biophysical rhythm 154 (e.g., a detected biophysical rhythm) of the person 101, as described with reference of FIG. 1.

The method 2300 further includes, at 2306, adjusting a playback speed of the audio data so that the audio data has a target playback tempo that matches a target biophysical rhythm, the target biophysical rhythm based at least in part on the detected biophysical rhythm. For example, the audio analyzer 140 of FIG. 1 adjusts a playback speed of the audio data 126 to the playback speed 134 so that the audio data 126 has the target playback tempo 162 that matches the target biophysical rhythm 164, as described with reference to FIG. 1.

The method 2300 thus automatically adjusts playback speed of the audio data 126, based at least in part on the biophysical sensor data 112. A technical advantage of the automatic playback speed adjustment can include the audio data 126 having the target playback tempo 162 that matches the biophysical rhythm 154 indicated by the biophysical sensor data 112. Listening to the audio signal 128 corresponding to the audio data 126 having the target playback tempo 162 can aid the person 101 in reaching or maintaining the target biophysical rhythm 164.

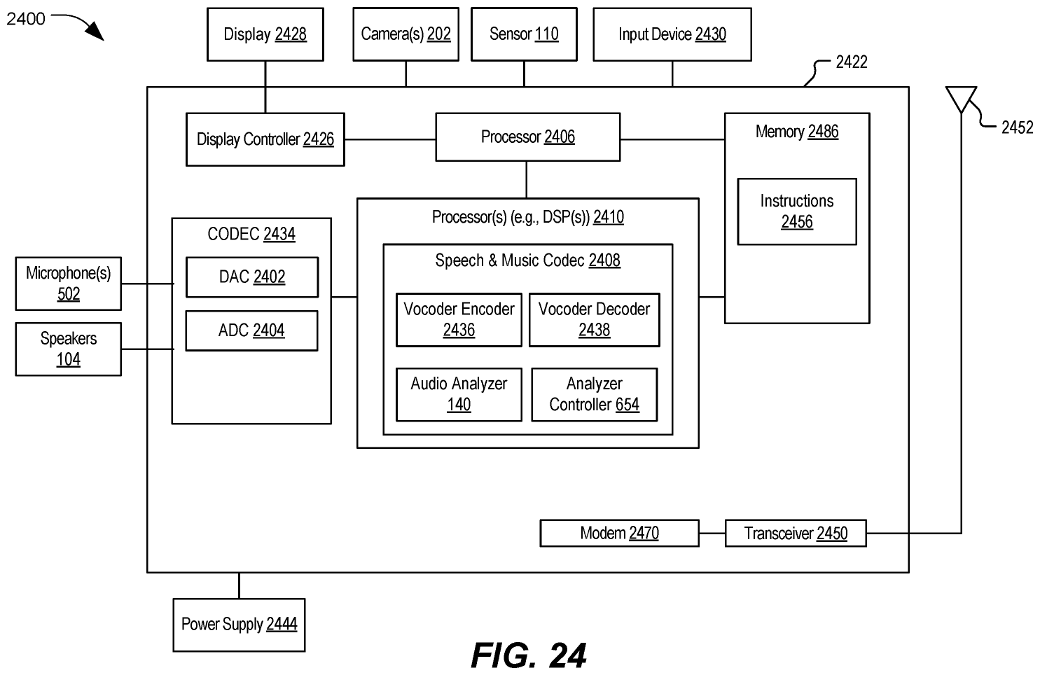

The method 2300 of FIG. 23 may be implemented by a field-programmable gate array (FPGA) device, an application-specific integrated circuit (ASIC), a processing unit such as a central processing unit (CPU), a digital signal processor (DSP), a controller, another hardware device, firmware device, or any combination thereof. As an example, the method 2300 of FIG. 23 may be performed by a processor that executes instructions, such as described with reference to FIG. 24.

Referring to FIG. 24, a block diagram of a particular illustrative implementation of a device is depicted and generally designated 2400. In various implementations, the device 2400 may have more or fewer components than illustrated in FIG. 24. In an illustrative implementation, the device 2400 may correspond to the device 102. In an illustrative implementation, the device 2400 may perform one or more operations described with reference to FIG. 1-23.

In a particular implementation, the device 2400 includes a processor 2406 (e.g., a CPU). The device 2400 may include one or more additional processors 2410 (e.g., one or more DSPs). In a particular aspect, the one or more processors 190 of FIG. 1 corresponds to the processor 2406, the processors 2410, or a combination thereof. The processors 2410 may include a speech and music coder-decoder (CODEC) 2408 that includes a voice coder (“vocoder”) encoder 2436, a vocoder decoder 2438, the analyzer controller 654, the audio analyzer 140, or a combination thereof.

The device 2400 may include a memory 2486 and a CODEC 2434. The memory 2486 may include instructions 2456, that are executable by the one or more additional processors 2410 (or the processor 2406) to implement the functionality described with reference to the audio analyzer 140, the analyzer controller 654, or both. The device 2400 may include a modem 2470 coupled, via a transceiver 2450, to an antenna 2452. In a particular aspect, the modem 2470 is configured to receive data via the transceiver 2450, and to provide the data to the processor 2406, the processors 2410, or a combination thereof. In some examples, the modem 2470 is configured to receive, via the transceiver 2450, at least some of the data that is processed or generated by the audio analyzer 140. In a particular aspect, the processor 2406, the processors 2410, or a combination thereof, are configured to provide data to the modem 2470, and the modem 2470 is configured to transmit the data via the transceiver 2450. In some examples, the modem 2470 is configured to transmit, via the transceiver 2450, at least some of the data that is processed or generated by the audio analyzer 140.

The device 2400 may include a display 2428 coupled to a display controller 2426. The one or more speakers 104, the one or more microphones 502, or a combination thereof, may be coupled to the CODEC 2434. The CODEC 2434 may include a digital-to-analog converter (DAC) 2402, an analog-to-digital converter (ADC) 2404, or both. In a particular implementation, the CODEC 2434 may receive analog signals from the one or more microphones 502, convert the analog signals to digital signals using the analog-to-digital converter 2404, and provide the digital signals to the speech and music codec 2408. The speech and music codec 2408 may process the digital signals, and the digital signals may further be processed by the analyzer controller 654, the audio analyzer 140, or both. In a particular implementation, the speech and music codec 2408 (e.g., the audio analyzer 140) may provide digital signals (e.g., the audio signal 128) to the CODEC 2434. The CODEC 2434 may convert the digital signals to analog signals using the digital-to-analog converter 2402 and may provide the analog signals to the one or more speakers 104.

In a particular implementation, the device 2400 may be included in a system-in-package or system-on-chip device 2422. In a particular implementation, the memory 2486, the processor 2406, the processors 2410, the display controller 2426, the CODEC 2434, and the modem 2470 are included in the system-in-package or system-on-chip device 2422. In a particular implementation, the sensor 110, the one or more cameras 202, an input device 2430, and a power supply 2444 are coupled to the system-in-package or the system-on-chip device 2422. Moreover, in a particular implementation, as illustrated in FIG. 24, the sensor 110, the one or more cameras 202, the display 2428, the input device 2430, the one or more speakers 104, the one or more microphones 502, the antenna 2452, and the power supply 2444 are external to the system-in-package or the system-on-chip device 2422. In a particular implementation, each of the sensor 110, the one or more cameras 202, the display 2428, the input device 2430, the one or more speakers 104, the one or more microphones 502, the antenna 2452, and the power supply 2444 may be coupled to a component of the system-in-package or the system-on-chip device 2422, such as an interface or a controller.

The device 2400 may include a smart speaker, a speaker bar, a mobile communication device, a smart phone, a cellular phone, a laptop computer, a computer, a tablet, a personal digital assistant, a display device, a television, a gaming console, a music player, a radio, a digital video player, a digital video disc (DVD) player, a tuner, a camera, a navigation device, a vehicle, a headset, an augmented reality headset, a mixed reality headset, a virtual reality headset, an aerial vehicle, a home automation system, a voice-activated device, a wireless speaker and voice activated device, a portable electronic device, a car, a computing device, a communication device, an internet-of-things (IoT) device, an extended reality (XR) device, a base station, a mobile device, or any combination thereof.

In conjunction with the described implementations, an apparatus includes means for obtaining audio data with a first playback tempo. For example, the means for obtaining can correspond to the audio analyzer 140, the one or more processors 190, the device 102, the system 100 of FIG. 1, the audio source 560, the input audio buffer 564, the tempo estimator 566, the tempo adjuster 568, the system 500 of FIG. 5A, the tempo based selector 556 of FIG. 5B, the mood based selector 558 of FIG. 5C, the processor 2406, the processors 2410, the modem 2470, the transceiver 2450, the device 2400, one or more other circuits or components configured to obtain audio data, or any combination thereof.

The apparatus also includes means for receiving biophysical sensor data indicative of a detected biophysical rhythm of a person. For example, the means for receiving can correspond to the audio analyzer 140, the one or more processors 190, the device 102, the system 100 of FIG. 1, the rhythm estimator 252 of FIG. 2, the target predictor 354 of FIGS. 3A-3B, the GCN 400 of FIG. 4A, the GCN 450 of FIG. 4B, the tempo adjuster 568, the system 500 of FIG. 5A, the rhythm combiner 954 of FIG. 9B, the processor 2406, the processors 2410, the modem 2470, the transceiver 2450, the device 2400, one or more other circuits or components configured to receive biophysical sensor data, or any combination thereof.

The apparatus further includes means for adjusting a playback speed of the audio data so that the audio data has a target playback tempo that matches a target biophysical rhythm, the target biophysical rhythm based at least in part on the detected biophysical rhythm. For example, the means for adjusting can correspond to the audio analyzer 140, the one or more processors 190, the device 102, the system 100 of FIG. 1, the tempo adjuster 568, the system 500 of FIG. 5A, the processor 2406, the processors 2410, the modem 2470, the transceiver 2450, the device 2400, one or more other circuits or components configured to adjust the playback speed, or any combination thereof.

In some implementations, a non-transitory computer-readable medium (e.g., a computer-readable storage device, such as the memory 2486) includes instructions (e.g., the instructions 2456) that, when executed by one or more processors (e.g., the one or more processors 2410 or the processor 2406), cause the one or more processors to obtain audio data with a first playback tempo (e.g., the playback tempo 152). The instructions, when executed by the one or more processors, also cause the one or more processors to receive biophysical sensor data (e.g., the biophysical sensor data 112) indicative of a detected biophysical rhythm (e.g., the biophysical rhythm 154) of a person (e.g., the person 101). The instructions, when executed by the one or more processors, further cause the one or more processors to adjust a playback speed (e.g., to the playback speed 134) of the audio data (e.g., audio data 126) so that the audio data has a target playback tempo (e.g., the target playback tempo 162) that matches a target biophysical rhythm (e.g., the target biophysical rhythm 164), the target biophysical rhythm based at least in part on the detected biophysical rhythm.

Particular aspects of the disclosure are described below in sets of interrelated Examples:

According to Example 1, a device includes: one or more processors configured to: obtain audio data with a first playback tempo; receive biophysical sensor data indicative of a detected biophysical rhythm of a person; and adjust a playback speed of the audio data so that the audio data has a target playback tempo that matches a target biophysical rhythm, the target biophysical rhythm based at least in part on the detected biophysical rhythm.

Example 2 includes the device of Example 1, wherein the target biophysical rhythm is the same as the detected biophysical rhythm.

Example 3 includes the device of Example 1 or Example 2, wherein the one or more processors are configured to predict the target biophysical rhythm based at least in part on the detected biophysical rhythm.

Example 4 includes the device of Example 3, wherein the one or more processors are configured to process, using a trained model, at least the detected biophysical rhythm to predict the target biophysical rhythm.

Example 5 includes the device of Example 4, wherein the trained model includes a graph convolutional network (GCN).

Example 6 includes the device of any of Example 3 to Example 5, wherein the one or more processors are configured to predict the target biophysical rhythm based on a time duration target, a calorie target, a user input, historical biophysical rhythm data, or a combination thereof.

Example 7 includes the device of any of Example 1 to Example 6, wherein the biophysical sensor data is received from a heart rate monitor.

Example 8 includes the device of any of Example 1 to Example, 7, wherein the detected biophysical rhythm corresponds to a heartbeat of the person.

Example 9 includes the device of any of Example 1 to Example 8, further including a heart rate monitor configured to: detect a heartbeat of the person; and generate the biophysical sensor data indicating the heartbeat as the detected biophysical rhythm.

Example 10 includes the device of any of Example 1 to Example 9, wherein the biophysical sensor data is received from one or more cameras.

Example 11 includes the device of any of Example 1 to Example 10, wherein the detected biophysical rhythm corresponds to a gait cadence of the person.

Example 12 includes the device of any of Example 1 to Example 11, further including one or more cameras configured to capture images of the person, the biophysical sensor data including the images, wherein the one or more processors are configured to process the images to estimate a gait cadence of the person as the detected biophysical rhythm.

Example 13 includes the device of any of Example 1 to Example 12, wherein the one or more processors are configured to, based on a comparison of the first playback tempo and the target playback tempo, obtain the audio data for adjustment.

Example 14 includes the device of any of Example 1 to Example 13, wherein the one or more processors are configured to, based on determining that a difference between the first playback tempo and the target playback tempo is within a difference threshold, obtain the audio data for adjustment.

Example 15 includes the device of any of Example 1 to Example 14, further including a camera configured to capture an image, wherein the one or more processors are configured to: process the image to determine a scene mood; and obtain the audio data based at least in part on determining that an audio mood of the audio data matches the scene mood.

Example 16 includes the device of Example 15, wherein the one or more processors are configured to determine the audio mood based on the first playback tempo, a music genre associated with the audio data, or both.

Example 17 includes the device of any of Example 1 to Example 16, wherein the one or more processors are configured to initiate playback, via one or more speakers, of an audio signal corresponding to the audio data having the adjusted playback speed.

Example 18 includes the device of Example 17, wherein the one or more processors are configured to: receive updated biophysical sensor data indicative of a change in the detected biophysical rhythm of the person; determine a second target biophysical rhythm based at least in part on the change in the detected biophysical rhythm; and initiate playback, via the one or more speakers, of an updated audio signal corresponding to second audio data having a second target playback tempo that matches the second target biophysical rhythm.

Example 19 includes the device of Example 18, wherein the one or more processors are configured to, in response to determining that a difference between the first playback tempo and the second target playback tempo is within a difference threshold, adjust the playback speed of the audio data to generate the second audio data.

Example 20 includes the device of Example 18, wherein the one or more processors are configured to, in response to determining that a difference between the first playback tempo and the second target playback tempo exceeds a difference threshold and that a difference between a second playback tempo of the second audio data and the second target playback tempo is within the difference threshold: obtain the second audio data having the second playback tempo; and adjust a playback speed of the second audio data so that the second audio data has the second target playback tempo.

Example 21 includes the device of any of Example 17 to Example 20, further including the one or more speakers configured to, during playback of the audio signal, output audio corresponding to the audio signal.

Example 22 includes the device of any of Example 17 to Example 21, further including a camera configured to capture a first image prior to playback of the audio signal, wherein the one or more processors are configured to: process the first image to determine whether a playback condition is detected; and based on determining that the playback condition is detected, initiate playback of the audio signal via the one or more speakers.

Example 23 includes the device of Example 22, wherein the camera is configured to capture a second image during playback of the audio signal, wherein the one or more processors are configured to: process the second image to determine whether a stop playback condition is detected; and based on determining that the stop playback condition is detected, discontinue playback of the audio signal via the one or more speakers.

Example 24 includes the device of any of Example 1 to Example 23, further including a modem configured to: receive second biophysical sensor data indicative of a second detected biophysical rhythm of a second person; and provide the second biophysical sensor data to the one or more processors, wherein the target biophysical rhythm is based on the second detected biophysical rhythm.

Example 25 includes the device of Example 24, wherein the one or more processors are configured to update the target biophysical rhythm to correspond to a combination biophysical rhythm that is based on the detected biophysical rhythm and the second detected biophysical rhythm.

Example 26 includes the device of any of Example 1 to Example 25, wherein the target biophysical rhythm is based on one or more additional detected biophysical rhythms of one or more additional persons.

According to Example 27, a method includes: obtaining, at a device, audio data with a first playback tempo; receiving, at the device, biophysical sensor data indicative of a detected biophysical rhythm of a person; and adjusting, at the device, a playback speed of the audio data so that the audio data has a target playback tempo that matches a target biophysical rhythm, the target biophysical rhythm based at least in part on the detected biophysical rhythm.

Example 28 includes the method of Example 27, wherein the target biophysical rhythm is the same as the detected biophysical rhythm.

Example 29 includes the method of Example 27 or Example 28, further including predicting the target biophysical rhythm based at least in part on the detected biophysical rhythm.

Example 30 includes the method of Example 29, further including processing, using a trained model, at least the detected biophysical rhythm to predict the target biophysical rhythm.

Example 31 includes the method of Example 30, wherein the trained model includes a graph convolutional network (GCN).

Example 32 includes the method of any of Example 29 to Example 31, further including predicting the target biophysical rhythm based on a time duration target, a calorie target, a user input, historical biophysical rhythm data, or a combination thereof.

Example 33 includes the method of any of Example 27 to Example 32, wherein the biophysical sensor data is received from a heart rate monitor.

Example 34 includes the method of any of Example 27 to Example, 33, wherein the detected biophysical rhythm corresponds to a heartbeat of the person.

Example 35 includes the method of any of Example 27 to Example 34, further including using a heart rate monitor to detect a heartbeat of the person; and generating the biophysical sensor data indicating the heartbeat as the detected biophysical rhythm.

Example 36 includes the method of any of Example 27 to Example 35, wherein the biophysical sensor data is received from one or more cameras.

Example 37 includes the method of any of Example 27 to Example 36, wherein the detected biophysical rhythm corresponds to a gait cadence of the person.

Example 38 includes the method of any of Example 27 to Example 37, further including: using one or more cameras to capture images of the person, the biophysical sensor data including the images, and processing the images to estimate a gait cadence of the person as the detected biophysical rhythm.

Example 39 includes the method of any of Example 27 to Example 38, further including, based on a comparison of the first playback tempo and the target playback tempo, obtaining the audio data for adjustment.

Example 40 includes the method of any of Example 27 to Example 39, further including, based on determining that a difference between the first playback tempo and the target playback tempo is within a difference threshold, obtaining the audio data for adjustment.

Example 41 includes the method of any of Example 27 to Example 40, further including: using a camera to capture an image; processing the image to determine a scene mood; and obtaining the audio data based at least in part on determining that an audio mood of the audio data matches the scene mood.

Example 42 includes the method of Example 41, further including determining the audio mood based on the first playback tempo, a music genre associated with the audio data, or both.

Example 43 includes the method of any of Example 27 to Example 42, further including initiating playback, via one or more speakers, of an audio signal corresponding to the audio data having the adjusted playback speed.

Example 44 includes the method of Example 43, further including: receiving updated biophysical sensor data indicative of a change in the detected biophysical rhythm of the person; determining a second target biophysical rhythm based at least in part on the change in the detected biophysical rhythm; and initiating playback, via the one or more speakers, of an updated audio signal corresponding to second audio data having a second target playback tempo that matches the second target biophysical rhythm.

Example 45 includes the method of Example 44, further including, in response to determining that a difference between the first playback tempo and the second target playback tempo is within a difference threshold, adjusting the playback speed of the audio data to generate the second audio data.

Example 46 includes the method of Example 44, further including, in response to determining that a difference between the first playback tempo and the second target playback tempo exceeds a difference threshold and that a difference between a second playback tempo of the second audio data and the second target playback tempo is within the difference threshold: obtaining the second audio data having the second playback tempo; and adjusting a playback speed of the second audio data so that the second audio data has the second target playback tempo.

Example 47 includes the method of any of Example 43 to Example 46, further including using the one or more speakers to, during playback of the audio signal, output audio corresponding to the audio signal.

Example 48 includes the method of any of Example 43 to Example 47, further including: using a camera to capture a first image prior to playback of the audio signal; processing the first image to determine whether a playback condition is detected; and based on determining that the playback condition is detected, initiating playback of the audio signal via the one or more speakers.

Example 49 includes the method of Example 48, further including: using the camera to capture a second image during playback of the audio signal; processing the second image to determine whether a stop playback condition is detected; and based on determining that the stop playback condition is detected, discontinuing playback of the audio signal via the one or more speakers.

Example 50 includes the method of any of Example 27 to Example 49, further including: using a modem to receive second biophysical sensor data indicative of a second detected biophysical rhythm of a second person; and providing the second biophysical sensor data to the one or more processors, wherein the target biophysical rhythm is based on the second detected biophysical rhythm.

Example 51 includes the method of Example 50, further including updating the target biophysical rhythm to correspond to a combination biophysical rhythm that is based on the detected biophysical rhythm and the second detected biophysical rhythm.

Example 52 includes the method of any of Example 27 to Example 51, wherein the target biophysical rhythm is based on one or more additional detected biophysical rhythms of one or more additional persons.

According to Example 53, a device includes: a memory configured to store instructions; and a processor configured to execute the instructions to perform the method of any of Example 27 to Example 52.

According to Example 54, a non-transitory computer-readable medium stores instructions that, when executed by a processor, cause the processor to perform the method of any of Example 27 to Example 52.

According to Example 55, an apparatus includes means for carrying out the method of any of Example 27 to Example 52.

According to Example 56, a non-transitory computer-readable medium stores instructions that, when executed by one or more processors cause the one or more processors to: obtain audio data with a first playback tempo; receive biophysical sensor data indicative of a detected biophysical rhythm of a person; and adjust a playback speed of the audio data so that the audio data has a target playback tempo that matches a target biophysical rhythm, the target biophysical rhythm based at least in part on the detected biophysical rhythm.

According to Example 57, an apparatus includes: means for obtaining audio data with a first playback tempo; means for receiving biophysical sensor data indicative of a detected biophysical rhythm of a person; and means for adjusting a playback speed of the audio data so that the audio data has a target playback tempo that matches a target biophysical rhythm, the target biophysical rhythm based at least in part on the detected biophysical rhythm.

Example 30 includes the apparatus of Example 29, wherein the means for obtaining, the means for receiving, and the means for adjusting are integrated into at least one of a smart speaker, a speaker bar, a smart phone, a cellular phone, a laptop computer, a computer, a tablet, a personal digital assistant, a display device, a television, a gaming console, a music player, a radio, a digital video player, a tuner, a camera, a navigation device, a vehicle, a headset, an augmented reality headset, a mixed reality headset, a virtual reality headset, an aerial vehicle, a car, a home automation system, a voice-activated device, a wireless speaker and voice activated device, a portable electronic device, a communication device, an internet-of-things (IOT) device, an extended reality (XR) device, a base station, or a mobile device.

Those of skill would further appreciate that the various illustrative logical blocks, configurations, modules, circuits, and algorithm steps described in connection with the implementations disclosed herein may be implemented as electronic hardware, computer software executed by a processor, or combinations of both. Various illustrative components, blocks, configurations, modules, circuits, and steps have been described above generally in terms of their functionality. Whether such functionality is implemented as hardware or processor executable instructions depends upon the particular application and design constraints imposed on the overall system. Skilled artisans may implement the described functionality in varying ways for each particular application, such implementation decisions are not to be interpreted as causing a departure from the scope of the present disclosure.

The steps of a method or algorithm described in connection with the implementations disclosed herein may be embodied directly in hardware, in a software module executed by a processor, or in a combination of the two. A software module may reside in random access memory (RAM), flash memory, read-only memory (ROM), programmable read-only memory (PROM), erasable programmable read-only memory (EPROM), electrically erasable programmable read-only memory (EEPROM), registers, hard disk, a removable disk, a compact disc read-only memory (CD-ROM), or any other form of non-transient storage medium known in the art. An exemplary storage medium is coupled to the processor such that the processor may read information from, and write information to, the storage medium. In the alternative, the storage medium may be integral to the processor. The processor and the storage medium may reside in an application-specific integrated circuit (ASIC). The ASIC may reside in a computing device or a user terminal. In the alternative, the processor and the storage medium may reside as discrete components in a computing device or user terminal.

The previous description of the disclosed aspects is provided to enable a person skilled in the art to make or use the disclosed aspects. Various modifications to these aspects will be readily apparent to those skilled in the art, and the principles defined herein may be applied to other aspects without departing from the scope of the disclosure. Thus, the present disclosure is not intended to be limited to the aspects shown herein but is to be accorded the widest scope possible consistent with the principles and novel features as defined by the following claims.

1. A device comprising:
one or more processors configured to:
obtain audio data with a first playback tempo;
receive biophysical sensor data indicative of a detected biophysical rhythm of a person; and
adjust a playback speed of the audio data so that the audio data has a target playback tempo that matches a target biophysical rhythm, the target biophysical rhythm based at least in part on the detected biophysical rhythm.

2. The device of claim 1, wherein the target biophysical rhythm is the same as the detected biophysical rhythm.

3. The device of claim 1, wherein the one or more processors are configured to predict the target biophysical rhythm based at least in part on the detected biophysical rhythm.

4. The device of claim 3, wherein the one or more processors are configured to process, using a trained model, at least the detected biophysical rhythm to predict the target biophysical rhythm.

5. The device of claim 4, wherein the trained model includes a graph convolutional network (GCN).

6. The device of claim 3, wherein the one or more processors are configured to predict the target biophysical rhythm based on a time duration target, a calorie target, a user input, historical biophysical rhythm data, or a combination thereof.

7-8. (canceled)

9. The device of claim 1, further comprising a heart rate monitor configured to:
detect a heartbeat of the person; and
generate the biophysical sensor data indicating the heartbeat as the detected biophysical rhythm.

10. The device of claim 1, wherein the biophysical sensor data is received from one or more cameras.

11. The device of claim 1, wherein the detected biophysical rhythm corresponds to a gait cadence of the person.

12. The device of claim 1, further comprising one or more cameras configured to capture images of the person, the biophysical sensor data including the images, wherein the one or more processors are configured to process the images to estimate a gait cadence of the person as the detected biophysical rhythm.

13. The device of claim 1, wherein the one or more processors are configured to, based on a comparison of the first playback tempo and the target playback tempo, obtain the audio data for adjustment.

14. The device of claim 1, wherein the one or more processors are configured to, based on determining that a difference between the first playback tempo and the target playback tempo is within a difference threshold, obtain the audio data for adjustment.

15. The device of claim 1, further comprising a camera configured to capture an image, wherein the one or more processors are configured to:
process the image to determine a scene mood; and
obtain the audio data based at least in part on determining that an audio mood of the audio data matches the scene mood.

16. The device of claim 15, wherein the one or more processors are configured to determine the audio mood based on the first playback tempo, a music genre associated with the audio data, or both.

17. The device of claim 1, wherein the one or more processors are configured to initiate playback, via one or more speakers, of an audio signal corresponding to the audio data having the adjusted playback speed.

18. The device of claim 17, wherein the one or more processors are configured to:
receive updated biophysical sensor data indicative of a change in the detected biophysical rhythm of the person;
determine a second target biophysical rhythm based at least in part on the change in the detected biophysical rhythm; and
initiate playback, via the one or more speakers, of an updated audio signal corresponding to second audio data having a second target playback tempo that matches the second target biophysical rhythm.

19. The device of claim 18, wherein the one or more processors are configured to, in response to determining that a difference between the first playback tempo and the second target playback tempo is within a difference threshold, adjust the playback speed of the audio data to generate the second audio data.

20. The device of claim 18, wherein the one or more processors are configured to, in response to determining that a difference between the first playback tempo and the second target playback tempo exceeds a difference threshold and that a difference between a second playback tempo of the second audio data and the second target playback tempo is within the difference threshold:
obtain the second audio data having the second playback tempo; and
adjust a playback speed of the second audio data so that the second audio data has the second target playback tempo.

21. (canceled)

22. The device of claim 17, further comprising a camera configured to capture a first image prior to playback of the audio signal, wherein the one or more processors are configured to:
process the first image to determine whether a playback condition is detected; and
based on determining that the playback condition is detected, initiate playback of the audio signal via the one or more speakers.

23. (canceled)

24. The device of claim 1, further comprising a modem configured to:
receive second biophysical sensor data indicative of a second detected biophysical rhythm of a second person; and
provide the second biophysical sensor data to the one or more processors, wherein the target biophysical rhythm is based on the second detected biophysical rhythm.

25-30. (canceled)
